(19) United States
(12) Patent Application Publication
(10) Pub. No.: US 2024/0245752 A1
(43) Pub. Date: Jul. 25, 2024

(54) METHODS FOR EFFECTIVE AND SAFE TREATMENT OF NEUTROPENIA USING DE-MOBILIZED G-CSF

(71) Applicant: TIANJIN PEGYLATT BIOTECHNOLOGY CO., LTD, Tianjin (CN)

(72) Inventor: Xuan Zhang, Tianjin (CN)

(73) Assignee: TIANJIN PEGYLATT BIOTECHNOLOGY CO., LTD, Tianjin (CN)

(21) Appl. No.: 16/538,853

(22) Filed: Aug. 13, 2019

(30) Foreign Application Priority Data
Aug. 5, 2019 (CN) 201910719064X

(51) Int. Cl. A61K 38/19; A61K 9/00; A61K 38/38; A61K 47/60; A61P 7/00

(52) U.S. Cl. CPC A61K 38/193; A61K 9/0019; A61K 38/38; A61K 47/60; A61P 7/00

(57) ABSTRACT

The present invention provides a method to treat and/or prevent febrile neutropenia and Grade 3/4 neutropenia. The method comprises application a Declined mobilizing granulocyte colony-stimulating factor (De-mobilized G-CSF) at a selected time, and a controlled dosage depended on the severity of neutropenia. The present invention also provides evidences to identify the De-mobilized G-CSF and High-mobilized G-CSF, and a method for preparing the De-mobilized G-CSF.

### FIELD OF THE INVENTION

The present invention relates to a new method for treating or preventing neutropenia by administrating a mobilizing-declined granulocyte colony-stimulating factor (De-mobilized G-CSF). The present invention further relates to identifying the De-mobilized G-CSF or the High-mobilized G-CSF among long-acting forms of G-CSF. The present invention also relates to a new method for site-specifically mono-modifying proteins or analogs thereof. In one aspect, the present invention relates to a method for N-terminally or C-terminally mono-pegylated G-CSF.

### BACKGROUND

Granulocyte colony stimulating factor (G-CSF) is a growth factor that promotes proliferation and differentiation of neutrophil progenitors, increases survival and activity of mature neutrophils, and involves in neutrophils release from bone marrow into circulation. Neutrophils, the most abundant white blood cells (WBC) in circulation, are a key player in host immune defense to fight bacterial and other infections. Neutrophils also influence the adaptive immune response.

Neutropenia is one of the most frequent and serious complications in cancer patients receiving myelosuppressive chemotherapy. Severe or Grade 3/4 neutropenia can lead to febrile neutropenia (FN), which is associated with significant morbidity and mortality. Both Filgrastim (marketed as Neupogen®), the first approved drug of rhG-CSF, and Pegfilgrastim (marketed as Neulasta®), the first approved long-acting form of rhG-CSF, are indicated to decrease the incidence of infection, as manifested by FN, in patients with non-myeloid malignancies receiving myelosuppressive anti-cancer drugs associated with a clinically significantly incidence of FN.

In clinical practice, Neulasta® is approved for 6 mg fixed dose and once in a cycle of chemotherapy (Once-per-cycle). Neupogen® is approved for 5 μg/kg/day daily for about 7-12 days. Both of them are administrated at 24 hours after chemotherapy to the patient with greater than 20% risk of FN. However, the severity and duration of chemotherapy-induced neutropenia (CIN) were significantly different among patients from with 20% to 60% risk of FN. For patients with greater than 40% predicted risk of FN, the approved methods for Pegfilgrastim and Filgrastim were shown to reduce only about 50% incidence of FN, and shorten the duration of Grade 3/4 neutropenia significantly, but overall in-hospital mortality rate of FN was still about 10%, which wasn't improved significantly compared with that of placebo control. There are also unmet medical needs for patients with less than 20% assessed risk of FN, whom were not recommended to use of Neulasta. Therefore, the clinical efficacy and safety of G-CSF-based therapy need to improve, and the therapeutic methods in treatment of severe neutropenia need to be more specific and individualized.

### SUMMARY

The invention provides a new method of application of a long-acting granulocyte colony-stimulating factor (“De-mobilized G-CSF”) to treat or prevent febrile neutropenia (FN) and/or Grade 3/4 neutropenia for a patient who suffers from chemotherapy-induced neutropenia (CIN). The method comprises that measuring an absolute neutrophils counts (ANC) and CD34⁺ cells counts in peripheral blood (PBPC) to obtain a response curve of neutrophils (ANC-RC) or progenitor cells (PBPC-RC) in a chemotherapy cycle, injecting a De-mobilized G-CSF to the patient at an administration time and at a dosage calculated based on a classified risk of FN and/or ANC determined on the planned injection day, to achieve an effective and safe ANC-RC (esANC-RC) and safe PBPC-RC (sPBPC-RC).

The long-acting G-CSF is a mobilizing-declined G-CSF, the De-mobilized G-CSF. The invention further provides evidences to identify the De-mobilized G-CSF, e.g., PEG30-rhG-CSF, and the High-mobilized G-CSF, a highly mobilizing G-CSF, among long-acting forms of G-CSF.

In some embodiments, the De-mobilized G-CSF is a kind of N-terminally linked G-CSF and characterized by: i) a PEG polymer conjugated or a Fc fragment or an albumin fused to the N-terminal of the G-CSF; ii) the molecular weight (MW) of the polymer or fusion protein is in range of 18,800-66,000 Dalton (18.8-66 kDa); iii) significantly increased CXCR4 expression comparing with G-CSF or C-terminally linked G-CSF; iv) significantly decreased the values of neutrophils and CD34⁺ cells release peaks comparing with rhG-CSF and High-mobilized G-CSF, e.g., G-CSF-C-P30.

The clinical therapeutic target of the new method for application of a De-mobilized G-CSF is to treat or prevent FN and Grade 3/4 neutropenia. The present invent has demonstrated that the clinical therapeutic target can be achieved through enhancing granulopoiesis with and by reducing mobilization of neutrophils and progenitor cells.

In some embodiments, the enhanced granulopoiesis and reduced mobilization in whole chemotherapy cycle can be represented by an effective and safe neutrophils response curve (esANC-RC) and a safe peripheral blood progenitor cells response curve (sPBPC-RC) by application of G-CSF products. The key parameters of esANC-RC for the enhanced granulopoiesis are that: i) the value of ANC nadir point is more than 1.5×10⁹ cells/L, preferably more than 2.0×10⁹ cells/L; ii) the value of ANC recovery peak is in the range of 5.0-15×10⁹ cells/L, preferably in the range of 5.0-10×10⁹ cells/L.

In some embodiments, the key parameters of esANC-RC for the reduced mobilization of the neutrophils are that: i) the value of ANC release peak is less than 24×10⁹ cells/L, preferably 12×10⁹ cells/L; and ii) the ratio of ANC release peak to ANC nadir point (P/B ratio) is less than 12, preferably less than 6.

In some embodiments, the key parameter of sPBPC-RC for the reduced mobilization of progenitor cells is that: the CD34⁺ cells count of the release peak in peripheral blood is less than 5×10⁶ cells/L, preferably less than 1×10⁶ cells/L.

In some embodiments, the standards of esANC-RC by application of a De-mobilized G-CSF consist of key parameters of the enhanced granulopoiesis and the reduced mobilization of neutrophils. In some embodiments, the standard of sPBPC-RC by application of a De-mobilized G-CSF consists of key parameter of the reduced mobilization of progenitor cells. In some embodiments, achieving the standards of esANC-RC and sPBPC-RC means to meet all the “preferably” values of ANC or CD34⁺ cells count as mentioned before.

In some embodiments, the therapeutic targets using the new method of the De-mobilized G-CSF are to meet standards of esANC-RC and sPBPC-RC. In some embodiments, a clinical therapeutic target is to entirely prevent the incidence of FN and/or Grade 3/4 neutropenia. The present invent has demonstrated that the clinical therapeutic target can be achieved by meeting standards of esANC-RC and sPBPC-RC.

In some embodiments, the administration time comprises a range of 3-9 days before the date of ANC nadir point. In some embodiments, the administration time is at the date after the neutrophil release peak induced by chemotherapy and/or when the ANC is back to the range of 1.5×10⁹-4.0×10⁹ cells/L.

In some embodiments, when the patient with CIN has a range of 10-40% assessed risk of FN, the administration time comprises about 5-7 days before the date of ANC nadir point, wherein the date of the ANC nadir point is between the Day 10 and Day 13 of the chemotherapy cycle.

In some embodiments, when the patient with CIN has a less than 10% assessed risk of FN and more than 50% risk of Grade 3/4 neutropenia, the administration time comprises about 3-5 days before the date of ANC nadir point.

In some embodiments, when the patient with CIN has a greater than 40% assessed risk of FN, the administration time comprises about 7-9 days before the date of ANC nadir point.

In some embodiments, when the patient with CIN who has a 10%-40% assessed risk of FN, or who preferentially has a not more than 20% assessed risk of FN, the dosage is not more than 50 μg/kg, preferably not more than 25 μg/kg, and the mode is Once-per-cycle.

In some embodiments, for the Once-per-cycle, the selected time and controlled dosage will be applied for the selected patient if the ANC determined on the planned injection day is in the range of 1.5-4.0×10⁹ cells/L. However, the controlled dosage will be increased to upper dose level of the mode if the ANC determined on the planned injection day is lower than 1.5×10⁹ cells/L; or the selected time will be postponed to the date when ANC determined returns in the range of 1.5-4.0×10⁹ cells/L, if the ANC determined on the planned injection day is more than 4.0×10⁹ cells/L; or the selected time will be postponed and/or the controlled dosage will be reduced to a lower dose level of the mode if the ANC determined on the planned injection day is higher than 8.0×10⁹ cells/L.

In some embodiments, when the patient with CIN has a less than 40% risk of FN, the single dose for 2-3 doses in a cycle is in a range of 10 μg/kg/dose-25 μg/kg/dose, and the dosing interval is three days, the mode is Every-3-day for 2 or 3 doses per cycle.

In some embodiments, when the patient with CIN has a greater than 40% assessed risk of FN, the single dose for 3-10 days in a cycle is in a range of 0.83 μg/kg/day-5 μg/kg/day, and the mode is Every-day.

In some embodiments, when the patient with has a greater than 20% assessed risk of FN, the administration time comprises one day after the chemotherapy. The cycle dosage is in a range of 10 μg/kg-50 μg/kg for lasting 3-7 days, and the mode is Continuing controlled delivery (CCD). In some embodiments, the CCD is performed by a micro-pump, a continuous delivery device, or a sustained release drug delivery system.

In some embodiments, all the modes selected display an ascending order of efficacy from Once-per-cycle, Every-3-day and Every-day to CCD based on the same cycle-dosage. In some embodiments, the mode applied can be switched to a more effective mode when FN or Grade 4 neutropenia occurs in the previous chemotherapy cycle. In some embodiments, the CCD mode can replace any of dose injection modes, e.g., Once-per-cycle, Every-3-days and Every-day, with significantly improved the on-target pharmacological effect and efficacy.

In some embodiments, if the ANC determined on the planned injection day is lower than 1.5×10⁹ cells/L, the dosage is administered at an upper dose level of the mode, and the dosage is in a range of 25 μg/kg-75 μg/kg or 1.5 mg-4.5 mg of the cycle-dosage.

In some embodiments, if the ANC determined on the planned injection day is higher than 8.0×10⁹ cells/L and/or if the administration time has been postponed for more than 4 days, the dosage is administered at a lower dose level of the mode, and the dosage is in a range of 10 μg/kg-50 μg/kg or 0.6 mg-3.0 mg of the cycle-dosage.

In some embodiments, the De-mobilized G-CSF comprises a kind of N-terminally linked G-CSF, e.g., PEG20-rhG-CSF, PEG30-rhG-CSF, PEG40-rhG-CSF, Fc fusion G-CSF, or albumin fusion G-CSF.

In one embodiment, one of the De-mobilized G-CSF is a kind of N-terminally mono-pegylated G-CSF, e.g., Pegfilgrastim and PEG30-rhG-CSF.

The N-terminally or C-terminally mono-pegylated G-CSF is a site-specifically mono-pegylated G-CSF. In one aspect, the invention provides a method for preparing the site-specifically mono-pegylated G-CSF, comprising i) mixing a cosolvent to a G-CSF solution to get a more than 4 mg/ml G-CSF solution; ii) adding an activated PEG polymer to the G-CSF solution, wherein the molar ratio of the PEG polymer to the G-CSF is in a range of 0.7-3 times, and under appropriate pH conditions for site-specific mono-conjugation of PEG on the N-terminal G-CSF or the C-terminal Thiol of G-CSF; iii) controlling a reaction time by keeping less than 5% of the formation rate of a di-pegylated G-CSF; wherein the 95% site-specific mono-conjugated G-CSF is homogenously N-terminally or C-terminally mono-pegylated G-CSF.

In some embodiments, the cosolvent is non-activated PEG, Bicine or sorbitol. In some embodiments, the pH for the N-terminal site-specific pegylation is about 3.0-5.0. In some embodiments, the pH for the C-terminal site-specific Thiol pegylation is about 7.0-8.5. In some embodiments, the site-specifically mono-pegylated method is also applied to site-specific conjugation of PEG polymers for a substrate peptide or a peptide for N-glycosylation of G-CSF analogs, or a carbonyl of sugar chain of G-CSF.

In some embodiments, the reaction time is monitored by high performance gel chromatography to control the formation rate of di-pegylated G-CSF less than 5% in conjugates mixer. In some embodiments, the N-terminally mono-pegylated G-CSF is a De-mobilized G-CSF, and C-terminally mono-pegylated G-CSF is a High-mobilized G-CSF.

In some embodiments, the conjugated PEG polymer is selected from PEG-ALD, PEG-SBA, PEG-SMB, PEG-SS, PEG-SG, PEG-NPC, PEG-isocyanate, or PEG-thiols, PEG-maleimides, PEG-OPSS, PEG-amine, PEG-hydrazide, PEG-aminoxy, PEG-alkyne or PEG-azide. In some embodiments, the PEG polymer is conjugated at N-terminal alfa amino acid or C-terminal free thiol group of amino acid of the G-CSF.

In some embodiments, the G-CSF comprises a fusion protein, e.g. albumin or Fc fragment at N-terminal or C-terminal of G-CSF. In some embodiments, the albumin or the Fc fragment comprises one or more domains or a variant. In some embodiments, the mutant further comprises one or more amino acid additions, deletions and/or substitutions, and properties of the albumin or Fc fragments or mutants may have an increased solubility, dimerization, affinity of FcRn or half-life as compared to the albumin or Fc fragment.

In some embodiments, the G-CSF comprises G-CSF fragments or G-CSF mutants. In some embodiments, the G-CSF mutant comprises one or more amino acid additions, deletions and/or substitutions, or small peptide fusion. In some embodiments, properties of G-CSF fragments or mutants comprise an increased solubility, stability, half-life and in vivo activity of the granulopoiesis, or a decreased activity of the mobilization, as compared to that of the G-CSF.

In some embodiments, the High-mobilized G-CSF is a kind of C-terminally linked G-CSF and characterized by: i) a PEG polymer or a Fc fragment or an albumin conjugated or fused to the C-terminal of the G-CSF; ii) the MW of the polymer or fusion protein is in range of 18.8-66 kDa; iii) significantly inhibited CXCR4 expression compared with De-mobilized G-CSF; iv) significantly increased the values of neutrophils and CD34⁺ cells release peaks compared with rhG-CSF and De-mobilized G-CSF, e.g., PEG30-rhG-CSF.

In some embodiments, the High-mobilized G-CSF is more suitable to apply for the indication of mobilization and collection of peripheral blood progenitor cells (PBPC).

### BRIEF DESCRIPTION OF THE DRAWINGS

FIG. 1 is a reproduction of the chromatogram of peaks from ion exchange chromatography of reaction mixture of pegylated rhG-CSF.

FIG. 2 is combined Western Blot that illustrates the effect of N-terminally or C-terminally linked G-CSF on CXC chemokine receptor-4 (CXCR4) expression in human CD33⁺ bone marrow myeloid cells.

FIG. 3A shows the effect of different administration times of PEG30-rhG-CSF or Neulasta on ANC response curves (ANC-RC) in rats with CIN.

FIG. 3B shows effects of the administration on Day 2 and on Day 3 of Neulasta on ANC-RC in rats with CIN. The administration on Day 3 of chemotherapy cycle is three days before the date of ANC nadir point.

FIG. 3C shows effects of different dose intervals for twice-per-cycle mode of PEG30-rhG-CSF on ANC-RC in rats with CIN. The minimized fluctuation of ANC-RC could be received by the Every-3-day, the three days dosing interval.

FIG. 3D shows effects of Every-3-day (90 μg/kg, twice) and Once-per-cycle (300 μg/kg or 600 μg/kg, once) of Neulasta on ANC-RC in rats with CIN. Standards of effective and safe ANC-RC (esANC-RC) were met by administration 90 μg/kg Every-3-day on Day 2 and Day 5 for Neulasta.

FIG. 3E shows effects of Every-3-day (60 μg/kg, twice) and Once-per-cycle (300 μg/kg or 600 μg/kg, once) of PEG30-rhG-CSF on ANC-RC in rats with CIN. Standards of esANC-RC were met by the mode of Every-3-day at single-dose of 60 μg/kg and the mode of Once-per-cycle of PEG30-rhG-CSF at cycle-dosage of 600 μg/kg.

FIG. 3F shows effects of Every-day (15 μg/kg/day for 5 days) and Once-per-cycle (150 μg/kg or 300 μg/kg, once) of PEG30-rhG-CSF on ANC-RC in rats with CIN. Standards of esANC-RC were met by the Every-day mode at a single-dose of 15 μg/kg/day for 5 days.

FIG. 3G shows effects of Every-day (30 μg/kg/day for 5 days, or 15 μg/kg/day for 10 days) and Every-3-Day (150 μg/kg, twice) of PEG30-rhG-CSF on ANC-RC in rats with CIN. More limited release peak of neutrophils was received by the Every-day mode at 15-30 μg/kg/day for 10 or 5 days, and higher neutrophils recovery peak was reached.

FIG. 3H shows effects of Every-day mode of rhG-CSF (50 μg/kg/day for 10 days) and Neulasta (5 μg/kg/day for 10 days) or PEG30-rhG-CSF (5 μg/kg/day for 10 days) on ANC-RC in rats with CIN. The key parameters of esANC-RC induced by single-dose of 5 μg/kg/day for PEG30-rhG-CSF or Pegfilgrastim are equivalent with or even better than that of single-dose of 50 μg/kg/day for rhG-CSF.

FIG. 4 shows effects of continuing controlled delivery for lasting three days (3-day-CCD, 120 μg/kg for 3 days), or for lasting five days (5-day-CCD, 120 μg/kg for 5 days) and Once-per-cycle (120 μg/kg, once, on Day 4) of PEG30-rhG-CSF or Pegfilgrastim on ANC-RC in macaques with CIN. Standards of esANC-RC were met by the 5-day CCD mode of PEG30-rhG-CSF and Pegfilgrastim.

FIG. 5 shows effects of different administration cycle-dosage and time of Once-per-cycle of Pegfilgrastim (50 μg/kg once on Day 7 and 100 μg/kg once on day 3) on ANC-RC for patients with CIN and with less than 10% predicted risk of FN. Standards of esANC-RC could be met by administrating Pegfilgrastim 50 μg/kg once on Day 7, not 100 μg/kg once on Day 3. FIG. 5 also shows effects of different administration single-dose of Filgrastim (2.5 μg/kg/day for 10 Days or 5 μg/kg/day for 10 days) on ANC-RC for the above CIN patients. Standards of esANC-RC could be met by administrating Filgrastim 2.5 μg/kg/day for 10 days, not 5 μg/kg/day for 10 days.

FIG. 6A shows effects of different cycle-dosages of Once-per-cycle (12.5, 25, 50, 75, 100 μg/kg once on Day 5) of PEG30-rhG-CSF on ANC-RC for patients with CIN and with about 20% predicted risk of FN. Key parameters of ANC-RC achieved by injection 50 μg/kg or 75 μg/kg of PEG30-rhG-CSF were closer to meet standards of esANC-RC.

FIG. 6B shows effects of different single-doses of Every-day mode for Filgrastim (2.5 μg/kg/day or g/kg/day from Day 5 to Day 14) on ANC-RC for patients with CIN and with about 20% predicted risk of FN. Key parameters of ANC-RC achieved by injection 2.5 μg/kg/day daily of Filgrastim were closer to meet standards of esANC-RC.

FIG. 6C shows effects of different modes and cycle-dosages of Once-per-cycle (12.5-100 μg/kg once on Day 5) of PEG30-rhG-CSF or single-doses of Filgrastim (1.25, 2.5 and 5 μg/kg/day for Day 5-14) on peripheral blood progenitor cells response curves (PBPC-RC) for patients with CIN and with about 20% predicted risk of FN. The standard of safe PBPC-RC (sPBPC-RC) was met by injection 50 μg/kg or less cycle-dosages once of PEG30-rhG-CSF in the selected patients.

FIG. 6D shows effects of different molecules, administration times and cycle-dosages of Once-per-cycle (50 or 100 μg/kg once on Day 5) of PEG30-rhG-CSF and Pegfilgrastim (60 or 100 μg/kg once on Day 2) on PBPC-RC for patients with CIN and with about 20% predicted risk of FN. The value of CD34⁺ cells release peak of PEG30-rhG-CSF 50 μg/kg or 100 μg/kg once on Day 5 was less than 75 times or 7 times that of Pegfilgrastim of 60 μg/kg or 100 μg/kg once on Day 2 in the selected patients.

FIG. 6E shows effects of different molecules and administration times of Once-per-cycle of PEG30-rhG-CSF (100 μg/kg once on Day 5) and Pegfilgrastim (100 μg/kg once on Day 2) on ANC-RC for patients with CIN and with about 20% predicted risk of FN. The value of ANC release peak of PEG30-rhG-CSF was significantly less than that of Pegfilgrastim, and the value of ANC nadir point was 4.3 times greater than that of Pegfilgrastim in the selected patients.

FIG. 7 shows effects of different molecules and administration modes, doses and times of Once-per-cycle of Pegfilgrastim (cycle-dosage 100 μg/kg once on Day 2, the approved method by FDA) or Every-day of Neupogen (single-dose 5 μg/kg/day) and Every-3-day of PEG30-rhG-CSF (single-dose 25 μg/kg twice on Day 3 and Day 6) on ANC-RC for patients with CIN and with equal to or greater than 40% predicted risk of FN. Standards of esANC-RC were met by the Every-3-day of PEG30-rhG-CSF. The value of ANC nadir point of PEG30-rhG-CSF was nearly 50 times greater than that of Pegfilgrastim, and the value of ANC release peak was 1.9 times less than that of Pegfilgrastim in the selected patients.

### DETAILED DESCRIPTION

As used herein, “homology” or “identity” or “similarity” refers to sequence similarity between two polypeptides or between two nucleic acid molecules. Homology can be determined by comparing one position or multi-positions in each of two sequences. The similarity of a polynucleotide or polynucleotide region (or a polypeptide or polypeptide region) can be represented as a certain percentage of bases (or amino acids) (for example, 60%, 65%, 70%, 75%, 80%, 85%, 90%, 95%, 98% or 99%) for “sequence identity” to another sequence when comparing the two sequences.

As used herein, the terms “treat” or “treatment” refer to both therapeutic treatment and prophylactic or preventative treatment, wherein the object is to cure or to prevent, or slow down (lessen) an undesired pathophysiological disorder, such as the neutropenia. Desired clinical results include, but are not limited to, cure or prevent disease, amelioration or palliation disease, stabilized the state of disease, delayed the progression of disease, whether detectable or undetectable. Those in need of treatment include those already with the disorder as well as those prone to have the disorder or those in which the disorder is to be prevented.

As used herein, the term “prevent” refers to administration or application of pharmaceuticals before the disease or disorder occurred. The “prevent” refers to prophylactic application or prophylactic treatment, wherein the object is to stop the disease or disorder, such as neutropenia.

As used herein, the term “neutrophils” refers to a type of white blood cell or granulocyte that protect us from infections. Neutrophils are the first cells to arrive on the site of a bacterial infection. Neutrophils address foreign invaders by a process referred to as phagocytosis, or as endocytosis. Neutrophils also help regulate the immune response in general.

As used herein, the term “ANC response curve” or “Neutrophils response curve” (ANC-RC) refers to dynamic records of ANC in an entire chemotherapy cycle. ANC is the absolute neutrophils count that reflects the count of mature neutrophils in circulation pool. Many factors including anti-cancer drugs and rhG-CSF products can lead to neutrophils response. ANC-RC reflects the daily count change of neutrophils in circulation on different days during a chemotherapy cycle. Several key parameters can represent the characteristics of ANC-RC, such as values of ANC nadir point, ANC release peak, ANC recovery peak, and ratio of ANC release peak to ANC nadir point et al.

As used herein, the term “response curve of progenitor cells in peripheral blood” (PBPC-RC) refers to dynamic records of CD34⁺ cells counts in circulation in a chemotherapy cycle. PBPC-RC reflects the values of release peak of progenitor cells or stem cells in peripheral blood, which are mobilized from bone marrow.

### Unrecognized Risks of Present G-CSF-Based Methods in Treatment of CIN

Two fundamental pharmacological properties, granulopoiesis and mobilization, were measured with the approved methods of Pegfilgrastim and Filgrastim in the treatment of patients with chemotherapy induced neutropenia (CIN).

Pegfilgrastim or Filgrastim could accelerate the granulopoiesis by stimulating the differentiation and proliferation of granulocyte progenitors, by shortening transit time, and by increasing absolute neutrophil count (ANC) in circulation. The value of ANC nadir point was elevated and its occurrence shifted towards the earlier date of the chemotherapy cycle, as compared in those receiving chemotherapy only. The recovery peak of neutrophils appeared 5-10 days after the administration of Pegfilgrastim or Filgrastim. This pharmacological property of G-CSF is called granulopoiesis, which exerts main on-target pharmacological effect in accelerating recovery of neutrophils after chemotherapy.

However, administration of Pegfilgrastim or Filgrastim could also promote the mobilization of mature neutrophils and progenitor cells or stem cells from bone marrow into peripheral blood. The neutrophils release peak lifts up at 4 hours after the injection, reaches the peak value at 12 hours and returns to a normal level within 4 days. The release peak of progenitor cells in peripheral blood is detected 5-8 days after the injection. This pharmacological property of G-CSF is called mobilization, which is main off-target pharmacological effect in accelerating recovery of neutrophils after chemotherapy.

For patients with solid tumors receiving cytotoxic chemotherapy, the approved methods of Pegfilgrastim and Filgrastim could cause huge release peaks of neutrophils and stem cells, which raise severe safety and efficacy concerns by present inventors.

The value of neutrophils release peak induced by 6 mg fixed-dose injection of Neulasta is about 32×10⁹ cells/L, which is 5 times higher than the up-limit of normal ANC. A large amount of valuable mature neutrophils are wasted during the early period of chemotherapy cycle. The numbers of the lost neutrophils could maintain normal ANC in peripheral blood for about 6 to 9 days in theory.

However, the huge neutrophils release peak do accelerate clearance of Pegfilgrastim in circulation. Evidences showed the receptor mediated clearance is the main eliminating mechanism for Pegfilgrastim, and other pegylated G-CSF e.g., PEG30-rhG-CSF or PEG40-rhG-CSF. The accelerated clearance of Pegfilgrastim in turn reduces the plasma half-life, bioavailability and efficacy of Pegfilgrastim.

An extremely high ANC may be one of possible causes leading to incidence of splenic rupture or acute respiratory distress syndrome, deadly complications for use of rhG-CSF products.

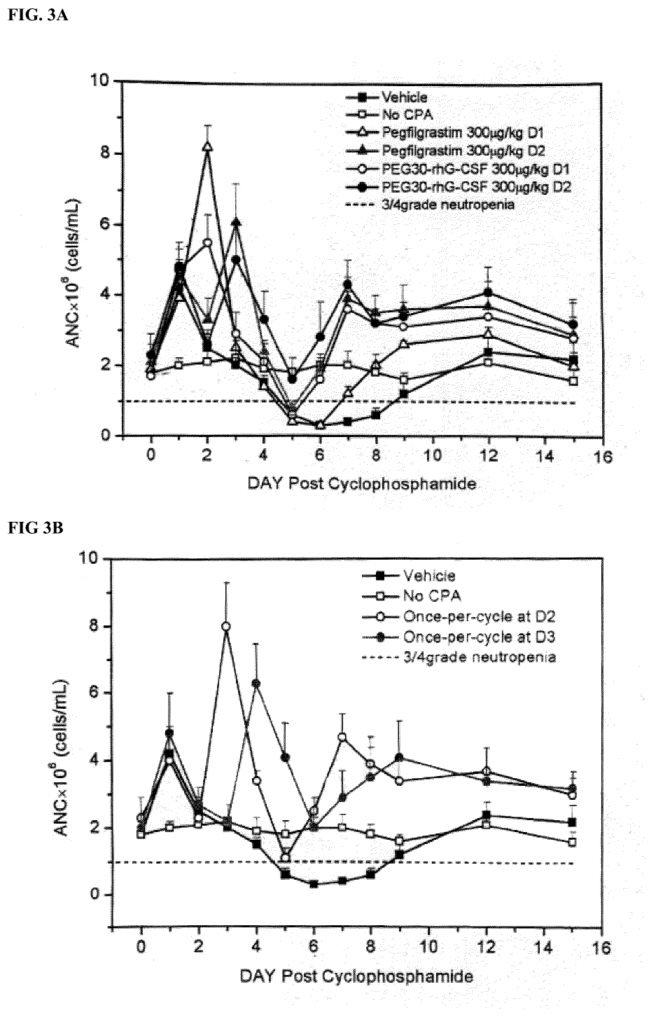

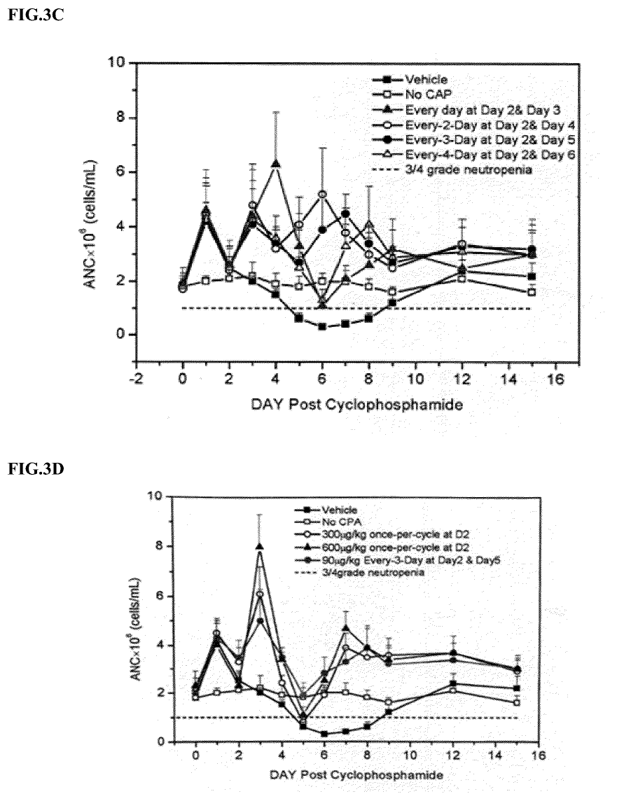

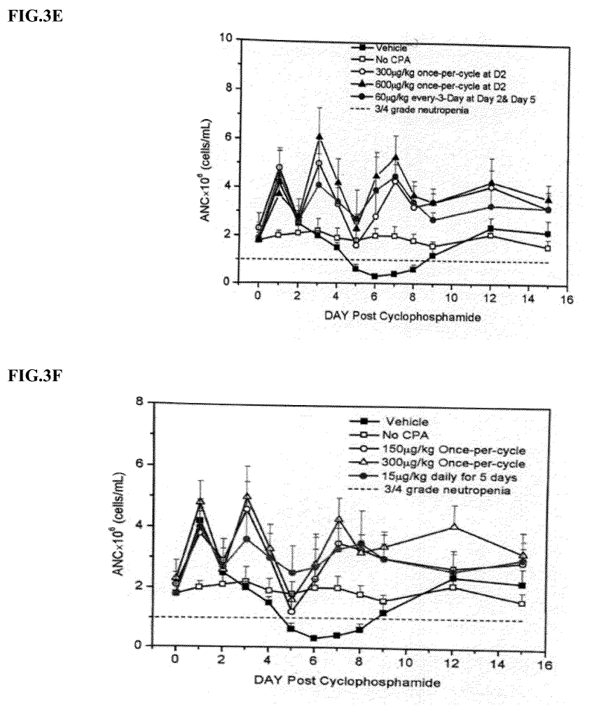

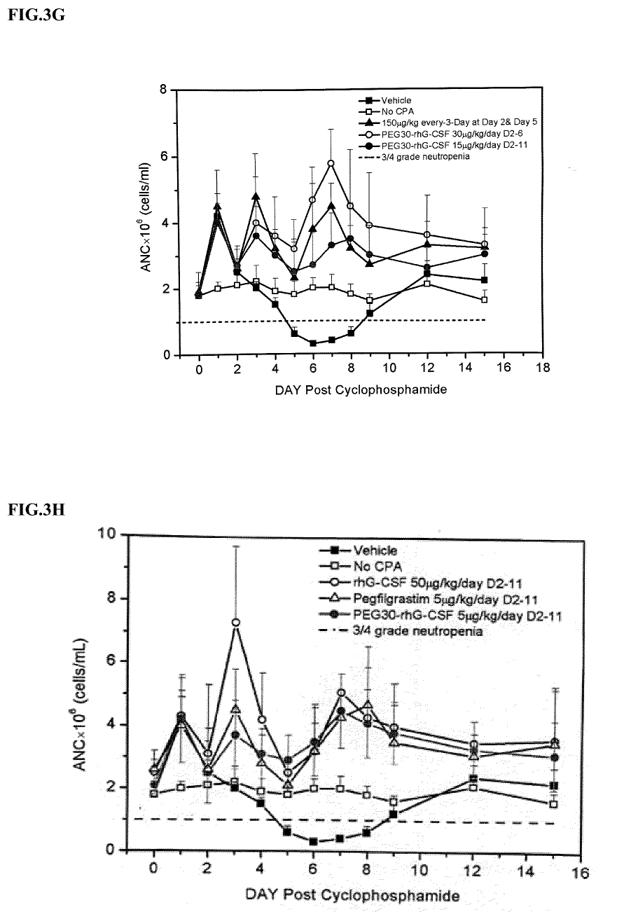

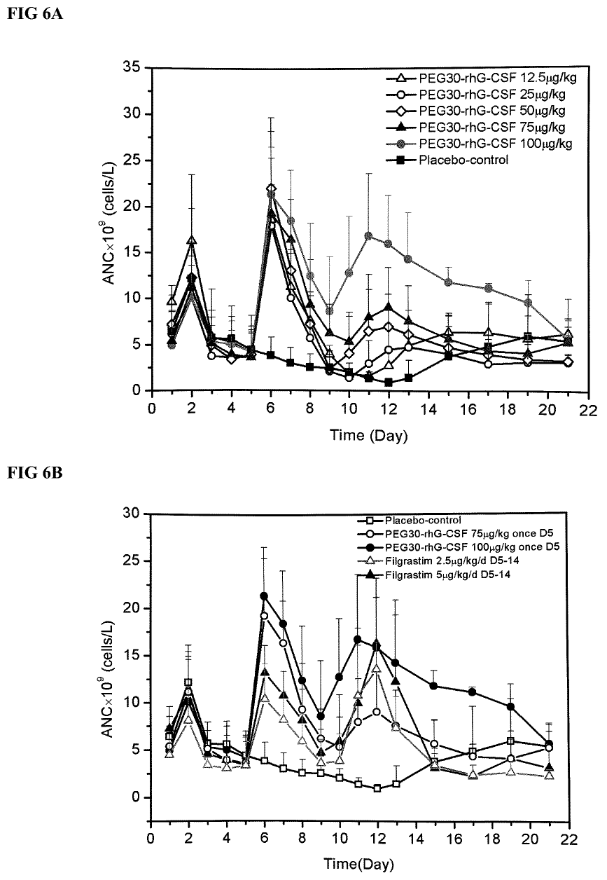

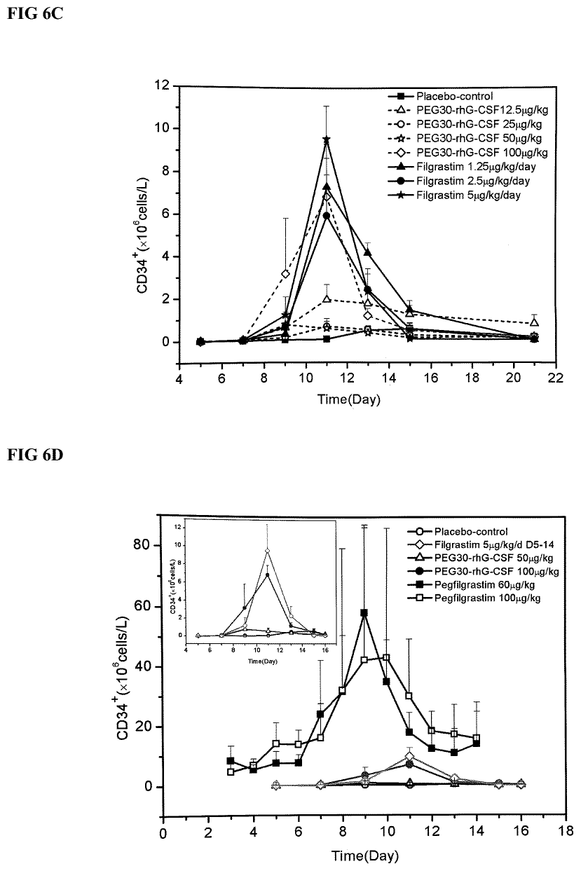

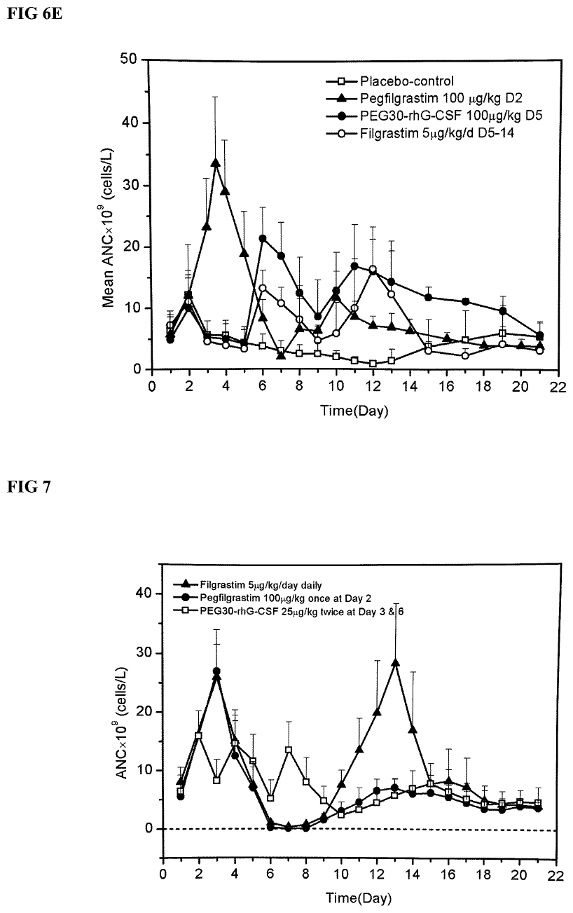

The neutrophils release peak results from accelerated clearance of neutrophils in storage pool of bone marrow. The instant inventors have identified that the ANC nadir point in chemotherapy cycle is a neutrophils release gap reflecting the survived mature neutrophils replaced by newly formed mature neutrophils. Importantly and unexpectedly, the present disclosure demonstrated that the extremely large amount of neutrophils released from bone marrow could lead to undesired or even deadly low and prolonged ANC nadir levels (as shown in FIGS. 3A, 3B, and 6A). And the function of neutrophils storage in bone marrow is severely damaged by the huge release peak.

Therefore, the huge neutrophils release peak is an unacceptable side effect or even a kind of toxicity for the clinical applications of both Pegfilgrastim and Filgrastim. This toxicity is an asymptomatic, ignored toxicity in clinical practice, because no ANC test is routinely monitored during the application of Pegfilgrastim.

Another and even greater safety risk associated with the approved methods of rhG-CSF products arises from their ability to mobilize progenitor cells or stem cells in bone marrow. Stem cells can be detected as CD34⁺ cells by flow cytometric analysis. After 4 days injection of Neulasta or Neupogen, the CD34⁺ cells counts could be monitored in peripheral blood, and CD34⁺ cells release peak could be detected on 6-7 days after the injection.

The mean count of CD34⁺ cells release peak induced by the 6 mg fixed dose injection of Pegfilgrastim is equal to that of induced by 10 μg/kg/day daily application of Filgrastim for 5-7 days. The CD34⁺ cells count for routine mobilization and collection of peripheral blood progenitor cells or stem cells (PBPC or PBSC) by injection 10 μg/kg/day daily of Filgrastim is equal to or more than 4×10⁷ cells/L, which is the standard for successful transplantation of PBPC or PBSC.

The mobilization of stem cells is a serious toxicity for patient with solid tumors receiving myelosuppressive anti-cancer drugs. The mobilization of CD34⁺ cells escalates damages of hematopoietic function of bone marrow induced by chemotherapy, and may lead to more sustained and severe neutropenia, more frequent risk of thrombocytopenia or even pancytopenia in continuous multiple cycles of chemotherapy. Thrombocytopenia is less frequent but higher dangerous complication of chemotherapy, which may result in deadly cerebral hemorrhage. This severe toxicity is also an asymptomatic, ignored toxicity in clinical practice, because CD34⁺ cells didn't be tested in treatment of CIN.

In summary, for patients with solid tumors receiving cytotoxic anti-cancer drugs, the approved methods of rhG-CSF products, in particular Pegfilgrastim, cause early appeared and/or huge release peaks of neutrophils and CD34⁺ cells, which raise severe safety and efficacy concerns not yet acknowledged in the art. Surprisingly and unexpectedly, however, the present disclosure demonstrates that a kind of N-terminally linked G-CSF have shown a declined mobilizing property, recognized as De-mobilized G-CSF, and by application of the De-mobilized G-CSF at selected administration times, controlled doses for present or innovative modes for CIN patients with classified risk of FN, the new method in treatment of CIN can (A) significantly enhance granulopoiesis, (B) substantially reduce the release peak of neutrophils, and (C) effectively reduce the release peak of CD34⁺ cells. In other words, the two fundamental pharmacological properties of G-CSF are remodeled, and the reduced mobilization of neutrophils and progenitor cells is one of mechanisms to enhance granulopoiesis. Further, new therapeutic targets and optimally clinical results that combine efficacy, safety and cost-effectiveness can be met by the remodeling.

### Remodeling the Pharmacologic Properties of G-CSF

In order to improve the clinical efficacy and safety for solid tumor patients with CIN, the granulopoiesis and mobilization of G-CSF should be dissociated to the opposite direction, which is to remodel the pharmacologic properties of G-CSF. The remodeling herein is to significantly enhance the granulopoiesis and substantially reduce mobilization of neutrophils and CD34⁺ cells.

In some embodiments, the remodeling in an entire chemotherapy cycle can be illustrated completely and accurately as an effective and safe ANC response curve (esANC-RC) and a safe peripheral-blood progenitor cells response curve (sPBPC-RC) by application of G-CSF products. In general, an ANC response curve (ANC-RC) is a dynamic, daily-based ANC records in a chemotherapy cycle, a peripheral-blood progenitor cells response curve (PBPC-RC) is a dynamic, daily-based CD34⁺ cells count records in a chemotherapy cycle. Therefore, new clinical therapeutic targets are to meet standards of esANC-RC and sPBPC-RC.

The present invent found standards of esANC-RC and sPBPC-RC could be characterized by some key parameters of the response curves. Present inventors conclude these key parameters of esANC-RC or sPBPC-RC based on clinical data. The therapeutic targets of new methods for De-mobilized G-CSF in treatment of CIN are to achieve these key parameters of esANC-RC or sPBPC-RC.

One of key parameters for enhanced granulopoiesis is the value of ANC nadir point, which is the only one therapeutic target by Neulasta. When the value of ANC nadir point is lower than 1.0×10⁹ cells/L, or even lower than 0.5×10⁹ cells/L, the patient becomes susceptible to infections, or has very high risk in suffering from FN, which is associated with significant morbidity and mortality.

Another key parameter for enhanced granulopoiesis is the value of ANC recovery peak. A high recovery peak of neutrophils may also be harmful to the hematopoietic function of the patient with multiple successive cycles of chemotherapy being performed. A controlled up-limit value of the ANC recovery peak means less granulocyte progenitors to be killed in the next chemotherapy cycle.

In some embodiments, key parameters of the esANC-RC for the enhanced granulopoiesis are that: i) the value of ANC nadir point is more than 1.5×10⁹ cells/L, preferably more than 2.0×10⁹ cells/L; ii) the value of ANC recovery peak is in the range of 5.0-15×10⁹ cells/L, preferably in the range of 5.0-10×10⁹ cells/L.

One of therapeutic targets is to reduce the ANC release peak to a reasonable controlled and safe level without delayed and/or with preferably increased ANC recovery. In one embodiment, a key parameter of the esANC-RC for the reduced mobilization is the value of neutrophil release peak should be significantly decreased as compared to that of the approved method of Pegfilgrastim. In another embodiment, one of objects for reducing ANC release peak is to elevate the value of ANC nadir point. Therefore, an index of the esANC-RC for the object be derived, a ratio of ANC release peak to value of ANC nadir point (P/B ratio). The ratio also reflects the fluctuation of the ANC-RC.

In some embodiments, the key parameters of the esANC-RC for the reduced mobilization of neutrophils are that: i) the up-limit value of ANC release peak is less than 24×10⁹ cells/L, or 18×10⁹ cells/L, and preferably 12×10⁹ cells/L; ii) the P/B ratio is less than 12, preferably less than 6.

One of therapeutic targets is to significantly reduce CD34⁺ cells release peak, the value of reduced CD34⁺ cells release peak should be significantly lower than that of the approved method of Pegfilgrastim and/or standard of CD34⁺ cells count for routine mobilization and collection of peripheral blood progenitor cells or stem cells (PBPC or PBSC).

In some embodiments, a key parameter of the sPBPC-RC for the reduced mobilization of progenitor cells is that the value of CD34⁺ cells release peak is not greater than 5×10⁶ cells/L, 4×10⁶ cells/L, 3×10⁶ cells/L, 2×10⁶ cells/L, or preferably not greater than 1×10⁶ cells/L.

Therefore, standards of an esANC-RC consist of key parameters of the enhanced granulopoiesis and the reduced mobilization of neutrophils. Both standards of esANC-RC and sPBPC-RC are therapeutic targets by application new methods of De-mobilized G-CSF in treatment of CIN.

In some embodiments, achieving the standards of esANC-RC and sPBPC-RC means to meet all the “preferably” values of ANC or CD34⁺ cells count of esANC-RC and sPBPC-RC as mentioned before.

In some embodiments, a clinical therapeutic target of the method of De-mobilized G-CSF in treatment of CIN is to entirely prevent the incidence of FN and/or Grade 3/4 neutropenia.

Importantly and unexpectedly, the present disclosure demonstrates that standards of esANC-RC and sPBPC-RC are significantly correlated to mortality rates in preclinical CIN model and clinical results of efficacy and safety as shown in Table 5&7. It is a surprised discovery that the approved method of Neulasta with high P/B ratio was harmful to the survival of the rat with CIN in successive three chemotherapy cycles. The mortality rate of the Neulasta application was significant increase compared with that of rats with CIN only. However, new methods of De-mobilized G-CSF (see in detail blow) that could meet standards of esANC-RC were beneficial to the survival of rats with CIN. If a new method of PEG30-rhG-CSF met standards of esANC-RC and sPBPC-RC, the patients with CIN were free of FN and Grade 3/4 neutropenia and no anti-infective use, so the clinical therapeutic target was achieved.

### De-mobilized G-CSF Molecules and Properties

The present invention has provided evidences to identify a declined mobilizing G-CSF or a highly mobilizing G-CSF among long-acting G-CSFs. The mobilizing declined G-CSF was named as De-mobilized G-CSF, which is a kind of N-terminally linked G-CSF, e.g., PEG30-rhG-CSF. The highly mobilizing G-CSF was named as High-mobilized G-CSF, which is a kind of C-terminally linked G-CSF, e.g., GCSF-C-P30. PEG30-rhG-CSF is one of N-terminally mono-pegylated G-CSF. GCSF-C-P30 is one of C-terminally mono-pegylated G-CSF.

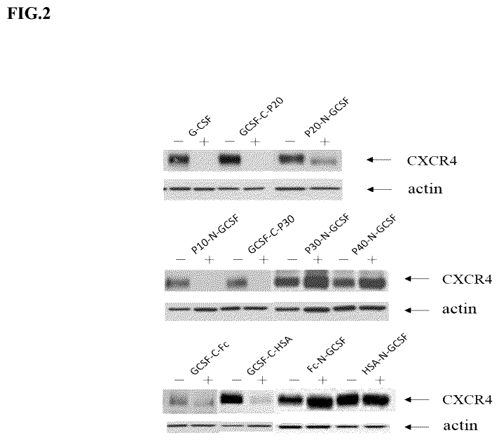

It is an unexpected and surprised discovery (as showed in Table 3) that release peaks of progenitor cells in peripheral blood (PBPC) induced by the N-terminally linked G-CSF are 3.5 to 14 times less than that of the C-terminally linked G-CSF, and also significantly less than that of rhG-CSF in health mice. The results indicated there were negative correlation between the mobilizing effects of PBPC and CXCR4 expression levels on stem cells as showed in FIG. 2.

As showed in Table 4, the values of ANC release peak induced by the N-terminally linked G-CSF are nearly two times less than that of the C-terminally linked G-CSF. The values of ANC nadir points induced by the N-terminally linked G-CSF are 2.8 to 11 times greater than that of the C-terminally linked G-CSF. And the values of ANC recovery peak induced by the N-terminally linked G-CSF are 1.4 to 3.6 times greater than that of the C-terminally linked G-CSF.

The N-terminally linked G-CSF has shown obviously reduced the mobilization of neutrophils and progenitor cells, and amplified the granulopoiesis compared with rhG-CSF and C-terminally linked G-CSF. Therefore, the N-terminally linked G-CSF in present invention is a declined mobilizing G-CSF, and recognized as the De-mobilized G-CSF.

The C-terminally linked G-CSF has shown obviously increased the mobilization of neutrophils and progenitor cells, and reduced the granulopoiesis compared with rhG-CSF and N-terminally linked G-CSF. Therefore, the C-terminally linked G-CSF in present invention is a highly mobilizing G-CSF, and recognized as High-mobilized G-CSF.

One of N-terminally linked G-CSF is the N-terminally mono-pegylated G-CSF. The polyethylene glycol (PEG) polymer can be conjugated to the N-terminal of the G-CSF; hence referred to as a PEG-rhG-CSF. The molecular weight (MW) of the PEG polymer is equal to or more than that of no-glycosylated rhG-CSF, which is 18.8 kDa, and in the range of 18.8 kDa-66 kDa. Preferably, the PEG polymer has a MW of about 30 kDa, or about 40 kDa, or from 30 kDa to 40 kDa. A non-limiting example of an N-terminally linked G-CSF is PEG30-rhG-CSF as tested in the experimental examples.

The PEG polymer can be linear or branched. Preferably, each G-CSF is conjugated to a single PEG polymer. In some embodiments, the PEG polymer is conjugated to the N-terminal amino acid residue of the protein. In some embodiments, the conjugation is at the N-terminal part of the G-CSF protein, or within the first 100, 50, 30, 20, 10, or 5 amino acid residues at the N-terminal. In some embodiments, the PEG polymer is conjugated to the sugar chain at the N-terminal of the protein.

One of N-terminally linked G-CSF is the Fc fragment or albumin fusion or conjugation to the N-terminal of G-CSF. The MW of Fc fragment or albumin is in the range of 18.8 kDa-66 kDa. The Fc fragments of antibody may be part of IgG, IgA, IgD, IgE or IgM. The Fc fragments may consist of whole of the Fc fragment or part of it, for example, consisting of CH2-CH3 domains or only CH3 domain. The Fc fragment or parts of it can be used as a dimmer or monomer. The sequences of Fc fragment or parts of it can be mutated to increase solubility, dimerization, affinity (e.g., FcRn, FcγRI, FcγRII) or half-life. The Fc fragment analogs can be used which include those having amino acid additions, deletions and/or substitutions. The albumin consists of 6 domains with about 66 kDa of MW in general. The albumin fused or conjugated may be a whole molecular of the albumin or one or several domains of it. The sequence of albumin or domains can be mutated to increase solubility, affinity (e.g., FcRn) or half-life.

The G-CSF of N-terminally linked G-CSF is a therapeutic protein used to treat neutropenia, infections, diabetes and tumors. The G-CSF or G-CSF mutant (G-CSFm) may be a glycosylated protein or a no-glycosylated protein, including an initial methionine amino acid residue (at position-1) of the G-CSF. The G-CSFm can include those having amino acid additions, deletions and/or substitutions.

The G-CSFm also can include N-terminal or initial fusion a substrate peptide, such as peptide AQQIVM for transglutaminase, or a peptide connector for glycosylation or pegylation, such as Asn-Xxx-Ser/Thr sequence for N-glycosylation and further glycol-pegylation, and ASGCGPE for Thiol pegylation. Mutation of G-CSF sequence can lead to reduce inhibitory activity against CXCR4 expression as compared to a wild-type human G-CSF, or to reduce in vivo activity of mobilization CD34⁺ cells from bone marrow to peripheral blood as compared to a wild-type human G-CSF. The mutation can be at one or more amino acid residues contemplated to impact such activity, such as those close to the N-terminal.

In some embodiments, the De-mobilized G-CSF is characterized by: i) a PEG polymer conjugated, or a Fc fragment or an albumin fused or conjugated, to the N-terminal of the G-CSF; ii) the MW of the PEG polymer or fusion protein is in range of 18,800-66,000 Dalton (18.8-66 kDa); iii) significantly increased CXCR4 expression comparing with G-CSF or C-terminally linked G-CSF; iv) significantly decreased the values of neutrophils and CD34⁺ cells release peaks comparing with rhG-CSF and High-mobilized G-CSF, e.g., G-CSF-C-P30.

In some embodiments, the High-mobilized G-CSF is characterized by: i) a PEG polymer conjugated, or a Fc fragment or an albumin fused or conjugated, to the C-terminal of the G-CSF; ii) the MW of the PEG polymer or fusion protein is in range of 18,800-66,000 Dalton (18.8-66 kDa); iii) significantly decreased CXCR4 expression comparing with G-CSF or N-terminally linked G-CSF; iv) significantly increased the values of neutrophils and CD34⁺ cells release peaks comparing with rhG-CSF and De-mobilized G-CSF, e.g., P30-N-G-CSF (PEG30-rhG-CSF).

In some embodiments, the present disclosure also demonstrates that the High-mobilized G-CSF can inhibit CXCR4 expression, and promote to mobilize CD34⁺ cells from the bone marrow to peripheral blood. In some embodiments, the High-mobilized G-CSF is more suitable to apply for the indication of mobilization and collection of peripheral blood progenitor cells (PBPC).

Meet Standards of esANC-RC and sPBPC-RC by New Methods of De-Mobilized G-CSF

The new molecules, De-mobilized G-CSF, have been identified to have superior capability to enhance granulopoiesis and reduce mobilization of neutrophils and progenitor cells. However, standards of esANC-RC and sPBPC-RC cannot be achieved by present approved administration time, dose, mode and the selected patients for Neulasta.

Importantly and unexpectedly, the present disclosure demonstrated that the significantly increased value of ANC nadir point or even removed the ANC nadir point can be achieved by reducing the neutrophils and stem cells release peak (as shown in FIG. 6E). And a limited reduction in value of the neutrophils release peak can lead to amplified increase in value of the ANC nadir point (as showed in Table 4, FIG. 3B, FIG. 6E).

The enhanced granulopoiesis and reduced mobilization can be achieved by a selected administration time and a controlled dosage for a patient with different severity of CIN. The selected administration time calculated based on classified risk of FN and/or the ANC determined on the planned injection day. The controlled dosage comprises a cycle-dosage or single-dose for Once-per-cycle, Every-3-day, Every-day and Continuing Controlled Delivery (CCD). The cycle-dosage is a total injection dose in a chemotherapy cycle. The single-dose is a single injection dose. The patients with CIN can be classified to three groups according to predicted risks of FN.

The present disclosure demonstrated that standards of esANC-RC and sPBPC-RC could be achieved by the selected administration time and the controlled dosage (as shown in Table 7, FIGS. 6C&6D and Example 5).

### Selected Admmstration Time

The suitable administration time can be selected by avoiding the irritated or reinforced neutrophils release. The size of mature neutrophils storage pool in bone marrow is also one of regulatory mechanisms on the level of the release peak.

Neutrophils release is a physiological response. Many drugs, e.g. anti-cancer drugs and corticosteroids, can trigger neutrophils release peak. To avoid irritating or reinforcing neutrophils release, the administration time of De-mobilized G-CSF should be selected on the date after the neutrophils release peak (as shown in FIGS. 3A & 6E), and/or on the date when ANC returns to the range of 1.5-4.0×10⁹ cells/L, which may be an indicator that the size of mature neutrophils storage pool in bone marrow is limited.

However, when there is no neutrophil release peak after administration of an anti-cancer drug, or evidence shows that the neutrophils storage pool in bone marrow is already depleted, the De-mobilized G-CSF could be injected 24 hours after chemotherapy. Therefore, ANC should be monitored at several key time points in chemotherapy cycle, including the pre-chemotherapy, 12 hours after chemotherapy, the planned injection day of De-mobilized G-CSF, 12 hours after the injection of De-mobilized G-CSF, and the date of ANC nadir point.

In general, the date of ANC nadir point occurred is related stable to all chemotherapy regimens for the patients with solid tumors, which can be measured or predicted by experienced professionals. In one embodiment, a suitable administration time is selected at least 3 days before the date of the ANC nadir point (as shown in FIGS. 3B & 5), which is the minimal duration for prophylactic application of De-mobilized G-CSF.

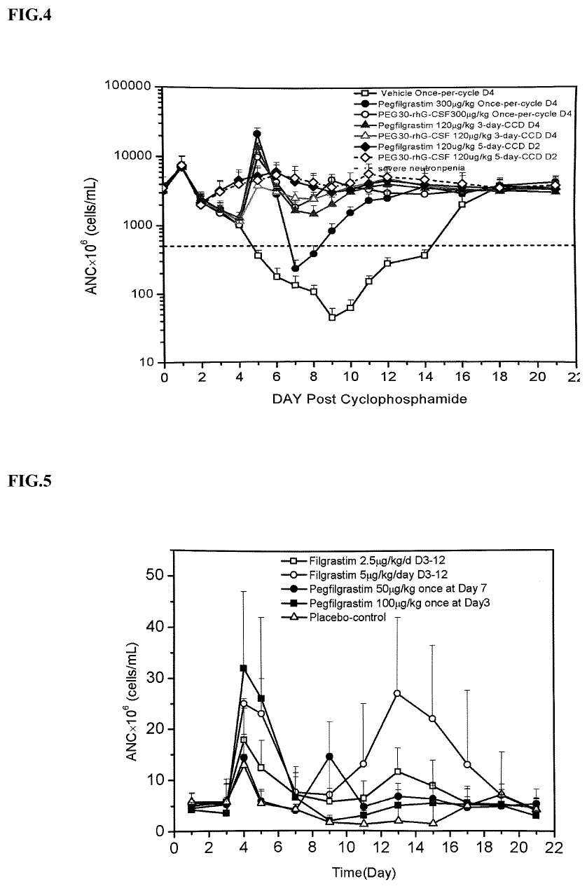

For instance, for a once-per-cycle mode, the administration time of a De-mobilized G-CSF can be on Day 3 (3 days before the date of ANC nadir point, as shown in FIG. 4), or on Day 5 (7 days before the date of ANC nadir point, as shown in FIG. 6A), or on Day 7 (4 days before the ANC nadir point, as shown in FIG. 5) of the chemotherapy cycle. Standards of esANC-RC could be achieved by all of these selected administration times instead of on Day 2 (10 days before the date of ANC nadir point), the approved administration time (as shown in FIGS. 3B & 5). The key parameters of ANC-RC induced by administration Neulasta on Day 2 or Day 3 were far inferior to standards of esANC-RC (as shown in FIGS. 5,6E &7).

More specifically, a selected administration time of De-mobilized G-CSF calculates based on classified risk of FN. For patients with CIN, when assessed risk of FN is in the range of 10%-40% (as shown in FIGS. 6A & 6B), the selected administration time is in the range of 5 to 7 days before the date of ANC nadir point; when assessed risk of FN is greater than 40%, the selected administration time is in the range of 7 to 9 days before the date of ANC nadir point (as shown in FIG. 7); when assessed risk of FN is less than 10%, and/or when the risk of Grade 3/4 neutropenia is more than 50% (as shown in FIG. 12), the selected administration time may be in the range of 3 to 5 days before the date of ANC nadir point (as shown in FIG. 5). Therefore, the administration time can be in the range of 3 to 9 days before the date of ANC nadir point.

In some embodiments, a selected administration time of De-mobilized G-CSF is based on the ANC determined on the planned injection day. When the ANC determined on the planned injection day is higher than 4.0×10⁹ cells/L, the administration time is postponed to the date when the ANC is dropped to the range of 1.5-4.0×10⁹ cells/L; When the ANC determined on the planned injection day is lower than 4.0×10⁹ cells/L, the administration time is at the same day.

In some embodiments, the selected administration time is suitable to all the first dose of single or multiple-doses modes of De-mobilized G-CSF.

### Controlled Dosage

In order to reduce mobilization of neutrophils and progenitor cells, the cycle-dosage and single-dose will be controlled for all modes applied. The mobilization of the PBPC depends mainly on the dosage and the molecule applied.

To reduce mobilization of progenitor cells, the cycle-dosage should be controlled to lower than 30 μg/kg for Pegfilgrastim when a single dose in a cycle (once-per-cycle mode) is administered (as shown in FIG. 6D). When Pegfilgrastim is administered as once every 3 days for two to three doses per cycle, the single-dose should be equal to or lower than 15 μg/kg. For daily administration, the single-dose should be equal to or lower than 5 μg/kg/day (as shown in Table 3). For continuing controlled delivery (CCD), the daily single-dose should be equal to or lower than 7 μg/kg/day for about 7 days, or the cycle-dosage for lasting 7 days delivery should be equal to or lower than 50 μg/kg (as shown in Table 6).

To reduce mobilization of progenitor cells, the cycle-dosage should be controlled to lower than 50 μg/kg for PEG30-rhG-CSF when a single dose per chemotherapy cycle is administered (as shown in FIG. 6D). When PEG30-rhG-CSF is administered as once every 3 days for two to three doses per cycle, the single-dose should be equal to or lower than 25 μg/kg (see in example 6). For daily administration, the single-dose should be equal to or lower than 5 μg/kg/day (as shown in Table 3). For CCD, the daily single-dose should be equal to or lower than 10 μg/kg/day for about 5 days, or the cycle-dosage for lasting 5 days delivery should be equal to or lower than 50 μg/kg (as shown in Table 6).

### Once-Per-Cycle Mode

The Once-per-cycle mode is a most simple and convenient mode for application of De-mobilized G-CSF, but the administration time, dosage, and indication should be carefully selected and controlled in order to meet standards of esANC-RC and sPBPC-RC.

In one embodiment, one of simplest way to improve the on-target efficacy of the Once-per-cycle mode is to control the cycle-dosage based on the ANC determined on the planned injection day. As shown in Example 5, when the ANC determined at 24 hours after chemotherapy is greater than 8.0×10⁹ cells/L, a controlled cycle-dosage should be reduced to a lower dose level and in the range of 0.3 mg-2.4 mg (5 μg/kg-40 μg/kg); and when the ANC determined at 24 hours after chemotherapy is lower than 1.5×10⁹ cells/L, a controlled cycle-dosage should be increased to an upper dose level and in the range of 0.6 mg-3 mg (10 μg/kg-50 μg/kg).

The present disclosure has demonstrated that the once-per-cycle mode will be more suitable to apply to patients with CIN when an assessed risk of FN is in a range of 10%-40%, preferably not more than 20%, and/or the duration of Grade 4 neutropenia lasting less than 5 days; and the administration time for the patient is on 5-7 days before the date of ANC nadir point; and the cycle-dosage is not more than 50 μg/kg.

In another embodiment, the cycle-dosage of Once-per-cycle is not more than 50 μg/kg, preferably not more than 25 μg/kg, and administration time on 3-5 days before the date of ANC nadir point can be used to patients with CIN when an assessed risk of FN is less than 10%, and/or the risk of Grade 3/4 neutropenia is more than 50%.

In order to achieve standards of the esANC-RC and sPBPC-RC, the controlled cycle-dosage should be selected to an upper dose level of the mode if the ANC determined on the planned injection day was lower than 1.5×10⁹ cells/L; and the controlled cycle-dosage should be selected to a lower dose level of the mode of if the ANC determined on the planned injection day was higher than 8.0×10⁹ cells/L and/or the planned injection time has been postponed for more than 4 days; or the selected time should be postponed if the ANC determined on the planned injection day is in the range of 4.0-8.0×10⁹ cells/L (as shown in Example 5).

Standards of the esANC-RC and sPBPC-RC can be achieved by utilizing Once-per-cycle of De-mobilized G-CSF with the new selected patients, time and dosage (as shown in Table 4, FIGS. 3F, 5, 6C &6D), and/or with administration time and/or dosage adjusted based on the ANC determined on the planned injection day (as shown in Table 7 and Example 5)

### Every-3-Day Mode

The present invent has discovered that an optimized dosing interval for multiple doses mode of De-mobilized G-CSF was not only related to the plasma clearance half-life, but also to the response trajectory of neutrophils in a chemotherapy cycle. Importantly and unexpectedly, the present disclosure demonstrated that the three-day dosing interval for 2-3 doses per cycle for De-mobilized G-CSF could minimize fluctuation of the ANC-RC compared with that of 4-or-more-day dosing intervals or 1-2 day dosing intervals (as shown in FIG. 3C). A minimized fluctuation of the ANC-RC was achieved by elevating the value of the ANC nadir point and reducing the value of the ANC release peak.

The new mode of three-day intervals is named herein “Every-3-day” (or “Every-3^{rd}-day”) Importantly, the significantly reduced release peaks of ANC and CD34⁺ cells can be monitored at single-dose lower than 25 μg/kg, specifically lower than 15 μg/kg (as shown in FIG. 6C), which is more than 6 times lower than the approved cycle-dosage of Pegfilgrastim. Every-3-day for De-mobilized G-CSF consists of two or three injection doses during a chemotherapy cycle depending on the duration of Grade 4 neutropenia.

The present disclosure has demonstrated that the Every-3-day mode could be applied to patients with CIN when an assessed risk of FN is equal to or less than 40%, and/or the duration of Grade 4 neutropenia lasting more than 5 days, and the first injection dose is on 5-7 days before the date of ANC nadir point occurred.

Standards of the esANC-RC and sPBPC-RC can be met by utilizing Every-3-day of De-mobilized G-CSF at dose in a range of 10-25 μg/kg (as shown in Table 6 and FIG. 6C & 6D). The fluctuation of ANC-RC and PBPC-RC for this mode was much less than that of Once-per-cycle at a cycle-dosage of 100 μg/kg (as shown in FIG. 3C-3E). The mobilization of CD34⁺ cells at doses of 10 μg/kg-25 μg/kg showed significant decrease by clinical trial.

### Every-Day Mode

Unexpectedly, further controlling the single-dose of De-mobilized G-CSF to as low as 5-30 μg/kg/day (0.83-5 μg/kg/day in human), and administration daily for 3-10 days could significantly elevate the value of ANC nadir point with significantly reduced mobilization of neutrophils and CD34⁺ cells (as shown in FIG. 3F-3H, Table 3). The effective cycle-dosage and single-dose of De-mobilized G-CSF for the daily mode, the Every-day mode, is lower and less than the approved cycle-dosage and single-dose of Filgrastim.

The less fluctuation of ANC-RC and equal to or higher value of ANC nadir point recorded by injecting 15 μg/kg/day for 5 days compared with that of Once-per-cycle at 300 μg/kg (as shown in FIG. 3F). More limited mobilization and higher neutrophils recovery peak were achieved by the Every-day mode at 15-30 μg/kg/day for 10 or 5 days compared with that of Every-3-days with 150 μg/kg, twice (as shown in FIG. 3G). No release peaks of stem cells and neutrophils could be detected by daily administering De-mobilized G-CSF 30 μg/kg/day for 5 days (as shown in Table 3). The results indicate that there is more effective potential for the Every-Day mode of De-mobilized G-CSF compared with the Once-per-cycle and Every-3-day modes. Therefore, Every-day mode could be applied to patient with CIN when an assessed risk of FN is a greater than 40%, and the first injection dose is on 7-9 days before the date of ANC nadir point occurred.

Importantly and surprisingly, to reach to the same level of ANC nadir point, the single-dose for daily administration of De-mobilized G-CSF can be less than 10 times that of the rhG-CSF with obviously reduced value of release peak of neutrophils (as shown in FIG. 3H). Therefore, the Every-day mode of De-mobilized G-CSF could replace Neupogen for almost all its indications approved except the mobilization and collection of PBPC.

As reported before, the minimal effective plasma concentration for Neulasta was about 2 ng/mL. The controlled single-dose for the daily mode is very low; the rhG-CSF concentration can be less than 2 ng/mL soon after the injection finished. Therefore, the Every-day mode of De-mobilized G-CSF can be easily applied to one-or-two-week durations of chemotherapy regimens and can be injected 24 hours after chemotherapy.

In some embodiments, when the ANC determined on the planned injection day is higher than 8.0×10⁹ cells/L, and/or when the administration date is postponed more than 4 days to reach the range of 1.5-4.0×10⁹ cells/L, the controlled dosage should be decreased to a lower dose level of the selected mode, and the cycle-dosage is in a range of 5 μg/kg-50 μg/kg or 0.3 mg-3.0 mg of the cycle-dosage.

In another embodiment, when the ANC determined on the planned injection day is lower than 1.5×10⁹ cells/L, the controlled dosage should be escalated to an upper dose level of the selected mode, and the cycle-dosage is in a range of 10 μg/kg-75 μg/kg or 0.6 mg-4.5 mg of the cycle-dosage.

### Continuing Controlled Delivery Mode (CCD)

Importantly and surprisingly, controlling the delivery rate of De-mobilized G-CSF by an injection pump can significantly reduce or even eliminate release peaks of ANC and CD34⁺ cells with the significantly increased value of ANC nadir point. Therefore, standards of esANC-RC and sPBPC are completely achieved by the CCD mode of De-mobilized G-CSF (as shown in FIG. 4 and Table 6).

The CCD mode is an innovative drug delivery mode for long-acting drugs like long-acting G-CSF. The combination of controlled drug delivery rate with the sustained duration of long-acting form of drugs in circulation dramatically improves the on-target pharmacological effect and efficacy of De-mobilized G-CSF. The fluctuation of the ANC-RC is kept in almost normal range of ANC and no CD34⁺ cells release peak detected.

When using the CCD mode, De-mobilized G-CSF can be administered 24 hours after anti-cancer drug application without co-reinforcing or stimulating ANC release peak. Continuing subcutaneous delivery of De-mobilized G-CSF can be controlled by insulin pumps or other continuous delivery devices, or a sustained release drug delivery system. The duration of continuous administration is in the range of 1 day to 10 days, preferentially from 3 to 7 days. The administration cycle dosages in CCD mode are in the range of 300 μg/cycle-3000 μg/cycle, or 5 μg/kg/cycle-50 μg/kg/cycle.

Standards of esANC-RC and sPBPC-RC could be met by the 120 μg/kg 3-day or 5-day CCD of De-mobilized G-CSF. Almost all ANC values in cycle were kept in normal range of 2.5-6.5×10⁹ cells/L with reduced or even disappeared neutrophils release peak (as shown in FIG. 4 and Table 6). The Once-per-cycle of Neulasta 300 μg/kg failed to meet standards of esANC-RC. No significant differences were found for the value of ANC release peak, ANC nadir point and P/B ratio between PEG30-rhG-CSF and Neulasta by controlled delivery rate of 24 μg/kg/day.

A dosage of 300 μg/kg for the macaque is equivalent to a dosage of 100 μg/kg for the human. Therefore, results showed that standards of the esANC-RC could be reached by 40 μg/kg (in human) CCD mode of De-mobilized G-CSF, more effective than that of 100 μg/kg Once-per-cycle mode in macaques with CIN; standards of sPBPC-RC could be meet by 50 μg/kg 5-Day-CCD of PEG30-rhG-CSF or 50 μg/kg 7-Day-CCD of Neulasta as shown on Table 6.

The CCD mode can deliver a higher cycle-dosage of De-mobilized G-CSF without reinforcing release peaks of neutrophils and progenitor cells compared with Once-per-cycle, Every-3-day and Every-day. All administration modes of De-mobilized G-CSF display an ascending order of efficacy from Once-per-cycle, Every-3-day and Every-day to CCD on the same cycle-dosage. Therefore, CCD mode could be applied to patient with CIN when an assessed risk of FN is a greater than 40%, and the dosage can be delivered 24 hours after chemotherapy.

CCD mode could be used for delivery of other long-acting drugs, such as albumin fusion G-CSF, Fc fusion G-CSF, albumin/Fc fusion-interferon, PEGylated interferon, albumin/Fc fusion-erythropoietin, PEGylated-erythropoietin, PEGylated-GLP-1, albumin/Fc fusion GLP-1, albumin/Fc fusion-human growth hormone, PEGylated-human growth hormone, PEGylated Fabs, antibodies, PEGylated Uricase and PEGylated Urate Oxidase, and their mutants PEGylated products.

### Prevention of Febrile Neutropenia and Grade 3/4 Neutropenia

The present technology can dramatically improve response curves of neutrophils and stem cells, standards of esANC-RC and sPBPC-RC can be achieved by selected a suitable administration time and a controlled dosage of a Demobilized G-CSF based on a classified risks of FN and the ANC determined on key time points in a chemotherapy cycle. Therefore, the Febrile Neutropenia (FN) and/or Grade 3/4 Neutropenia can be prevented completely.

The incidence of FN in patients with CIN positively correlates to the severity and duration of neutropenia. A chemotherapy regimen with more than 60% assessed risk of FN means that more than 90% incidence of Grade 4 neutropenia will be occurred. However, a chemotherapy regimen with about 20% assessed risk of FN means that more than 60% incidence of grade 3 or lower grade neutropenia will be occurred. Neulasta with the fixed dose and time isn't suitable for all those patients.

Therefore, the present disclosure divides the CIN into three subclasses according to classified the assessed risk of FN: i) the first group: >40% risk of FN; ii) the second group: 10%-40% risk of FN; and iii) the third group: <10% risk of FN and/or >50% risk of Grade 3/4 neutropenia.

In one embodiment, in order to prevent FN and/or Grade 3/4 neutropenia, the cycle-dosage for Once-per-cycle mode was controlled to not more than 50 μg/kg; the selected patients were the patients with CIN who have 10-40% risk of FN; and the selected administration time was on 5-7 days before the date of ANC nadir point.

In another embodiment, the cycle-dosage for Once-per-cycle mode was controlled to not more than 50 μg/kg, preferably not more than 25 μg/kg; the selected patients were patients with the third group of CIN; and the selected administration time was on 3-5 days before the date of ANC nadir point. Standards of esANC-RC could be achieved, and no incidence of Grade 3/4 neutropenia was occurred as shown in FIG. 5.

Therefore, Once-per-cycle mode was for the patient with CIN who has 10%-40%, preferentially not more than 20% predicted risk of FN, and the cycle-dosage for Once-per-cycle mode was controlled to not more than 50 μg/kg, preferably not more than 25 μg/kg.

In some embodiments, adjustable dosage based on the determined ANC for Once-per-cycle is one of simplest ways to improve the ANC-RC of present approved method of Neulasta. Patients with CIN and with 10%-40% risk of FN were selected. The initially controlled and planned cycle-dosage for PEG30-rhG-CSF is 40 μg/kg and the selected administration time on Day 2 of the chemotherapy cycle. The cycle-dosage of 40 μg/kg was injected when the ANC determined on Day 2 was in the range of 1.5-8.0×10⁹ cells/L. The cycle-dosage could be increased to 50 μg/kg if the ANC determined on Day 2 was lower than 1.5×10⁹ cells/L. The cycle-dosage could be reduced to 30 μg/kg if the ANC determined on Day 2 was higher than 8.0×10⁹ cells/L. Standards of esANC-RC could be almost achieved by simply adjustable dosage (as shown in Example 5).

In some embodiments, adjustable dosage and/or time based on the ANC determined on the planned injection day for Once-per-cycle were applied for the CIN patients with assessed 10%-40% risk of FN. An initially controlled cycle-dosage for PEG30-rhG-CSF is 25 μg/kg and the planned injection time on Day 5 of the chemotherapy cycle, which is 7 days before the date of ANC nadir point. The cycle-dosage of 25 μg/kg was injected when the ANC determined on Day 5 was 1.5-4.0×10⁹ cells/L. The administration dosage can be increased to 50 μg/kg if the ANC determined on Day 5 was lower than 1.5×10⁹ cells/L. However, if the ANC determined on Day 5 is in the range of 4.0-8.0×10⁹ cells/L, the administration time can be postponed to the date when the ANC determined is in the range of 1.5-4.0×10⁹ cells/L. And if the ANC determined on Day 5 is higher than 8.0×10⁹ cells/L and/or the ANC returns to the range of 1.5-4.0×10⁹ cells/L is postponed more than 4 days after the selected date, the administration dosage can be reduced to a lower dose level of the mode, such as 12.5 μg/kg. Standards of esANC-RC and sPBPC could be achieved, and no incidence of Grade 3/4 neutropenia was occurred as shown in Table 7.

The Every-3-Day mode was carried out in treatment of CIN patients with assessed risk of FN not more than 40% and/or the duration of Grade 4 neutropenia lasting more than 5 days. The controlled single-dose of Every-3-Day for two or three doses per chemotherapy cycle was in a range of 10 μg/kg-25 μg/kg, and the selected injection time was on 5-7 days before the date of ANC nadir point. Three doses in a cycle were applied for the patient with the duration of Grade 4 neutropenia lasting more than 5 days.

The second and/or third doses can be increased to upper dose level of the mode, such as 25 μg/kg of single-dose if the ANC determined on the planned injection time was lower than 1.5×10⁹ cells/L. Standards of esANC-RC and sPBPC-RC can be achieved by this new mode, and the value of ANC nadir point was much greater than that of Once-per-cycle mode at a cycle-dosage of 100 μg/kg (as shown in FIG. 7).

The application of Every-Day with single-dose in a range of 0.83-5 μg/kg/day could be carried out in treatment of patients with CIN and with greater than 40% assessed risk of FN, and the selected administration time was on 7-9 days before the date of ANC nadir point. Standards of esANC-RC and sPBPC-RC could be met by the daily mode. Evidences in preclinical CIN mode showed that less fluctuation of ANC-RC and higher value of ANC nadir point were achieved by 2.5 μg/kg/day for 5 days compared with that of once-per-cycle at 50 μg/kg. More limited mobilization and higher neutrophils recovery peak were achieved by the Every-day mode at 0.83-5 μg/kg/day for 10 or 5 days compared with that of Every-3-days (25 μg/kg, twice).

For CCD mode of De-mobilized G-CSF, the rate of subcutaneous absorption is controlled and can be slower than that of subcutaneous injection modes. So higher cycle dosage can be delivered without accelerating neutrophils or CD34⁺ cells release. The effective cycle-dosage of CCD mode for PEG30-rhG-CSF is 10 times lower than that of Once-per-cycle for Neulasta in term of standards of esANC-RC and sPBPC-RC (as shown in Table 6).

In one embodiment, the application of CCD mode with cycle-dosage in range of 10 μg/kg-50 μg/kg for 3-7 days can be applied for the treatment of patients with CIN who have a greater than 40% assessed risk of FN.

In one embodiment, the application of CCD mode with cycle-dosage in the range of 5 μg/kg-10 μg/kg can be employed in treatment of patients with CIN and with less than 40% or between 20%-40% risk of FN. Therefore, the application of CCD mode with cycle-dosages in range of 5 μg/kg-50 μg/kg or 300 μg/cycle-3000 μg/cycle for 3-7 days can be employed in treatment of a CIN patient with more than 20% assessed risk of FN, and/or the duration of Grade 4 neutropenia lasting more than 5 days.

In one embodiment, an administration time for CCD mode could be 24 hours after chemotherapy or Day 1-Day 3 of chemotherapy cycle. In some embodiments, the CCD can be performed by a micro-pump, a continuous delivery device, or a sustained release drug delivery system.

The CCD mode of De-mobilized G-CSF could replace any of dose injection modes, e.g. once-per-cycle, Every-3-days and Every-day, with significantly improved on-target pharmacological effect and efficacy of De-mobilized G-CSF. In some embodiments, all of said modes display an ascending order of clinical efficacy from Once-per-cycle, Every-3-day and Every-day to CCD on the same cycle-dosage. In some embodiments, a more effective mode can be applied for present chemotherapy cycle should when FN or Grade 4 neutropenia occurs in the previous chemotherapy cycle.

Every-day mode and CCD mode of De-mobilized G-CSF can be applied to all indications of rhG-CSF or Filgrastim except one, the mobilization of PBPC. The Every-day and CCD mode of De-mobilized G-CSF could replace the Every-day mode of rhG-CSF or Filgrastim with significantly improved on-target pharmacological effect and efficacy of the rhG-CSF or Filgrastim.

Every-Day or CCD mode of De-mobilized G-CSF can replace rhG-CSF or Filgrastim in treatment for all patients including Cancer Patients Receiving Myelosuppressive Chemotherapy, Patients with Acute Myeloid Leukemia Receiving Induction or Consolidation Chemotherapy, Cancer Patients Receiving Bone Marrow Transplant, Engraftment following the mobilized Peripheral Blood Progenitor Cells (PBPC) in Cancer Patients and Patients with Severe Chronic Neutropenia.

Every-Day or CCD mode of De-mobilized G-CSF can be used in multiple one- or two-week of chemotherapy regimens for treatment of CIN. It is because the G-CSF concentration in circulation can be eliminated to 2 ng/ml or lower in a short period of time.

### Preparation of Site-Specific Mono-Pegylated G-CSF

The invention provides a method for preparing a site-specific mono-pegylated G-CSF, comprising i) adding a cosolvent to a G-CSF solution to get a more than 4 mg/ml G-CSF solution; ii) adding an activated PEG polymer to a concentrated G-CSF solution, wherein the molar ratio of the PEG polymer to the G-CSF is in the range of 0.7-3.0 times, and a site-specific PEG conjugation reaction on the N-terminal amino G-CSF or the C-terminal site-specific Thiol group of G-CSF under appropriate pH conditions; iii) controlling the formation rate of a di-pegylated G-CSF to be less than 5% in a mixer; wherein the site-specific mono-conjugated G-CSF is homogenously 95% or more N-terminally or C-terminally mono-pegylated G-CSF.

The present disclosure provides a new method to modify a protein by activated Polyethylene glycol (PEG) polymers in a site-specific and mono-pegylated way. In general, for the pegylated reaction, the molar ratio of PEG polymer to protein molecule can be up to or higher than several or several dozens of times, the reaction rates increase, and so do the multiple-pegylated proteins simultaneously, if there are several reaction groups in the protein molecule. In a different aspect, the present disclosure provides a method for producing a site-specific mono-pegylated protein by increasing molar ratios of protein to activated PEG molecule. The site-specific and mono-modification rates significantly increase with relatively lower molar ratios of PEG polymer to protein molecules, longer reaction time and under suitable pH condition.

G-CSF solution was concentrated by ultrafiltration. The concentration of the G-CSF solution was in the range of 4-20 mg/ml. The cosolvents in the present method were Bicine, non-activated PEG polymers and sorbitol. One of the activated PEG polymers was methoxy-PEG with a single active aldehyde (mPEG-ALD) or methoxy-PEG with a single active maleimide (mPEG-MAL) for the site-specific Thiol pegylation. One of the site-specific modifications was N-terminal amino or C-terminal Thiol group of amino acid of the protein. The prolonged reaction duration was about 12-36 hours.

One of the suitable conditions for site-specific N-terminal pegylation was a pH range of 2.0-6.0, preferably in the range of 3.0-5.0. One of the suitable conditions for site-specific Thiol pegylation was a pH range of 5.5-8.5, preferably in the range of 6.5-8.5.

Site-specific modification and mono-pegylated rhG-CSF means that only one PEG polymer and one protein conjugate at the site-specific position of the protein. The activated PEG polymers, which attach to the N-terminal amino group of protein, also includes mPEG-n-hydroxysuccinimidyl ester (mPEG-SCM), mPEG-succinimidyl propionate (mPEG-SPA), mPEG-succinimidyl butanoate (mPEG-SBA), mPEG-succinimidyl a-methylbutanoate (mPEG-SMB), mPEG-succinimidyl succinate (mPEG-SS), mPEG-succinimidyl glutarate (mPEG-SG), mPEG-p-nitrophenyl carbonate (mPEG-NPC) and mPEG-isocyanate. The activated PEG polymers, which attach to the free Thiol group of amino acid of the protein, also include mPEG-maleimides, mPEG-ortho-pyridyldisulfide (mPEG-OPSS), mPEG-vinyl sulfone (mPEG-VS) and mPEG-thiols. The activated PEG polymers that could link to N-terminal G-CSF also include PEG-alkyne and PEG-azide for “click” conjugation, and PEG-amine, PEG-hydrazide and PEG-aminoxy for carbonyl conjugation. The molecular weight (MW) of activated PEG polymer is in a range of 18.8 kDa˜66 kDa. The proteins also include interferons, erythropoietin, Glucagon like peptide-1 (GLP-1), human growth hormone, Fab, uricase or urate oxidase and their mutants.

### Methods for Preparation of N-Terminally Fused G-CSF

The Fc fusion proteins, e.g., Fc-N-GCSF, have been constructed so that N-terminal of G-CSF was linked to the C-terminal of the IgGl-Fc fragment. The amino acid sequence of IgGl-Fc fragment was shown in SEQ ID NO.2. Fc-N-GCSF was expressed and secreted by CHO cells, and purified by Protein A affinity chromatography and further by size exclusion chromatography. On a molar basis, the Fc-N-GCSF fusion protein had biological activities comparable to that of G-CSF in a G-CSF-dependent in vitro cell proliferation assay. The half-lives of Fc fusion proteins in rats were about 24 hours, 10 times longer than that of G-CSF.

The albumin fusion proteins, e.g., HSA-N-GCSF, have been constructed so that the N-terminal of G-CSF is linked to the C-terminal of the human serum albumin. The amino acid sequence of human serum albumin is seen in UniProtKB-P02768 (ALBU_HUMAN). HSA-N-GCSF was expressed and secreted by CHO cells, and purified by ion exchange chromatography and further by size exclusion chromatography. On a molar basis, the HSA-N-GCSF fusion proteins had biological activities that were 10 times lower than that of G-CSF in a G-CSF-dependent in vitro cell proliferation assay. The half-lives of albumin fusion proteins in rats were about 56 hours, more than 20 times longer than that of G-CSF.

### Example 1. Production and Characteristic of N-Terminally Linked G-CSF

1. Method for Production of rhG-CSF

The genetically engineered expression plasmids containing the human G-CSF gene or G-CSF mutant genes were routinely constructed according to methods of molecular cloning and expression. The amino acid sequence of human G-CSF (hG-CSF) was shown in UniProtKB-P09919 (CSF3_HUMAN). The expression plasmid with a hG-CSF or hG-CSF mutant gene was transformed into an Escherichia coli (E. coli) bacteria to construct a genetically engineered G-CSF high expression bacteria (G-CSF engineered bacteria).

Recombinant hG-CSF (rhG-CSF) was produced by fermentation of the G-CSF engineered bacteria in a 15-liter BIOSTAT C fermenter (B. Braun, Germany). The bacteria pellets were collected by centrifugation 5000 rpm for 30 minutes at 4° C. (Sorvall RC3B, DuPont, USA), re-suspended with TE buffer (50 mM Tris, 1 mM EDTA, pH 7.5) and then disrupted by high-pressure homogenizer (APV 2000, APV, USA). The broken bacteria material was centrifuged at 11000 rpm for 20 minutes at 4° C. (Sorvall RC5B, DuPont, USA). The pellets were collected, re-suspended the inclusion body (IB) pellets with washing solution (50 mM Tris, 1 mM EDTA, 2.5% Triton X100, pH7.5), and stirred, then centrifuged at 11000 rpm for 50 minutes; repeated the procedure three times to isolate the purified IB. The IB was dissolved in denaturing solution (50 mM Tris, 1 mM EDTA, 8M guanidine hydrochloride, 10 mg/mL Dithioerythritol, pH 8.0) at 4° C. for 12 hours. The IB denaturing solution was centrifuged at 11000 rpm for 40 minutes to collect supernatant. The supernatant was slowly added to the refolding buffer (20 mMTris, 1 mM EDTA, 0.5M Urea, pH 8.5, 0.2 g/L oxidized glutathione), and diluted 50-100 times, kept at 4° C. for 24 hours. The refolded rhG-CSF solution was loaded to ion exchange column (SP Sepharose FF, GE Healthcare, USA) to separate rhG-CSF. The rhG-CSF protein was eluted and then further purified with size exclusion chromatography (Superdex 75, GE Healthcare, USA) to obtain purified rhG-CSF solution (20 mM sodium acetate, pH 4.5).

### 2. Method for Production of G-CSF Fusion Proteins

The genes of recombinant fusion proteins, e.g., Fc-N-GCSF, GCSF-C-Fc, HSA-N-GCSF or GCSF-C-HSA, had been constructed to form either N-terminal of G-CSF linked to the C-terminal of the IgG-Fc fragment (Fc-N-GCSF), or C-terminal of G-CSF linked to the N-terminal of the IgG-Fc fragment (GCSF-C-Fc) via a modified hinge of EPKSSDKTHTAP. So did the HSA-N-GCSF or GCSF-C-HSA. The amino acid sequence of IgGl-Fc fragment was shown in UniProtKB-P01857 (IGHG1_HUMAN). Fc-N-GCSF or GCSF-C-Fc was expressed and secreted by CHO cells, and purified by Protein A affinity and size exclusion chromatography. The in vitro activity of Fc-N-GCSF or G-CSF-C-Fc was 2-5×10⁷IU/mg determined by the NFS60 cell proliferation assay. On a molar basis, the in vitro activity of Fc-N-GCSF or G-CSF-C-Fc was comparable to that of G-CSF. The half-live of two fusion proteins in rats was about 24 hours, 10 times longer than that of G-CSF.

The amino acid sequence of human serum albumin was shown in UniProtKB-P02768 (ALBU_HUMAN). HSA-N-GCSF or GCSF-C-HSA was expressed and secreted by CHO cells, and purified by ion exchange chromatography and further by size exclusion chromatography. The in vitro activity of HSA-N-GCSF or G-CSF-C-HSA was 5-20×10⁵ IU/mg determined by the NFS60 cell proliferation assay. On a molar basis, the in vitro activity of HSA-N-GCSF or G-CSF-C-HSA was 10 times lower than that of G-CSF. The half-live of two fusion proteins in rats was about 56 hours, more than 20 times longer than that of G-CSF.

3. Method for Pegylation of N-Terminally or C-Terminally Site-Specifically Mono-Pegylated rhG-CSF

The rhG-CSF solution (20 mM sodium acetate, pH 4.5) was added non-activated PEG polymer at a molar ratio of 3:1 (PEG: rhG-CSF), and then concentrated to 4 to 6 mg/ml. The methoxypolyethylene glycol propionaldehyde (mPEG-ALD, NOF, Japan) with average molecular weight of 30000 Daltons (30 kDa) and sodium cyanoborohydride were added to the concentrated rhG-CSF solution at a molar ratio of 2.5:1 (mPEG-ALD: rhG-CSF). The pH of the reaction system was 3.5-5.0. The reaction mixture was stirred at 4° C. for 12-16 hours. Monitored the reaction mixer by High-pressure liquid chromatography (HPLC) with a Biosep S2000 column; and when the formation rate of a di-pegylated G-CSF was near 5%, added 20 times volume of 1 mM HCl solution (pH 4.0) in the mixer to terminate the reaction, and got a finalized reaction mixer.

A 10 mg/ml solution of rhG-CSFmCl8A&P175C in 100 mM Bicine (pH 7.5) was added to solid mPEG-maleimide (methoxy polyethylene glycol maleimide) (JenKem, China) with average molecular weight of 30 kDa. A 1.5 molar excess mPEG-maleimide to rhG-CSF was used. The pH of the reaction system was 7.0-8.5. The reaction mixture was stirred under 4° C. for 3-5 hours. Monitored the reaction mixer by HPLC with Biosep 52000 column; and when the formation rate of a di-pegylated G-CSF is near 5%, added 10 times volume of sterile water in the mixer to terminate the reaction; adjusted the pH to 4.0 with diluted HCl, and got a finalized reaction mxier.

### 4. Method for Purification of N-Terminally or C-Terminally Mono-Pegylated G-CSF

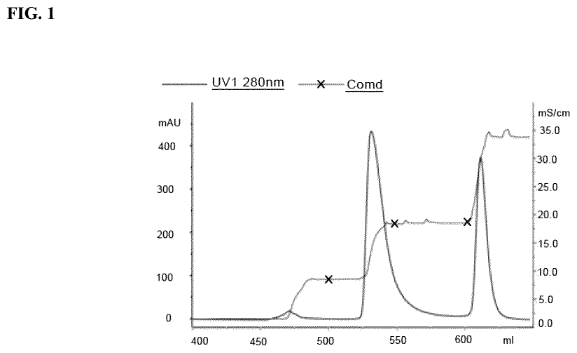

The N-terminally or C-terminally mono-pegylated G-CSF was purified by ion exchange chromatography (SP Sepharose FF,) in AKTA purifier 100 (GE Healthcare, USA). The finalized reaction mixtures were loaded to the SP Sepharose FF column. The rhG-CSF and pegylated rhG-CSF were separated by gradient elution with mixering buffer A (10 mM Sodium Acetate, pH 4.0) and buffer B (10 mM Sodium Acetate and 1M NaCl pH 4.0), and the gradient was from 8% buffer B to 20% buffer B then to 40% buffer B. FIG. 1 was a reproduction of the chromatogram of peaks from ion exchange chromatography of finalized reaction mixture of N-terminally mono-pegylated G-CSF. Three peaks were showed in FIG. 1, the first peak was a mixer contained di-pegylated rhG-CSF and mono-pegylated rhG-CSF, the second peak was mono-pegylated rhG-CSF, and the third peak was free rhG-CSF, which was further confirmed by analysis of HPLC and SDS-PAGE electrophoresis (data not provided).

As shown in Table 1, a ratio of the site-specifically mono-pegylated rhG-CSF was increased with concentration of rhG-CSF in a range of 4.0-6.0 mg/ml, and higher ratio could be reached with a lower molar ratio of PEG-ALD to rhG-CSF in a high concentrated G-CSF solution.

TABLE 1
Effect of rhG-CSF concentration on ratios of site-specifically mono-pegylated rhG-CSF

| rhG-CSF concentration | 4.0 mg/ml | 5.0 mg/ml | 6.0 mg/ml | 4.0 mg/ml | 10 mg/ml | 20 mg/ml |
|---|---|---|---|---|---|---|
| Activated PEG polymers | mPEG-ALD | mPEG-ALD | mPEG-ALD | mPEG-ALD | mPEG-OPSS | mPEG-ALD |
| Molar ratio of PEG:rhG-CSF | 2.5:1 | 2.5:1 | 2.5:1 | 5:1 | 1.5:1 | 0.8:1 |
| Mono-PEG30-rhG-CSF^{a} | 55.7% | 67.0% | 76.3% | 63.5% | 68.8% | 65.3% |
| Di-PEG30-rhG-CSF | 4.3% | 11.1% | 18.4% | 13.9% | 7.2% | 4.9% |
| Free rhG-CSF | 40.0% | 21.9% | 5.3% | 22.6% | 24.0% | 29.8% |

^{a}”PEG30” is represented a mPEG-ALD with 30,000 Da (30 kDa) molecular weight.

5. Pharmacokinetic Parameters of Pegylated rhG-CSF

Pharmacokinetic parameters of Pegylated rhG-CSF with different molecular weights for patients with CIN were shown in Table 2 (see in detail at example 5). The results showed that the plasma clearance half-life (t_{1/2}) of PEG30-rhG-CSF was almost twice as long as that of PEG20-rhG-CSF, and there was little difference between t_{1/2} of PEG30-rhG-CSF and t_{1/2} of PEG40-rhG-CSF.

TABLE 2
Clinical Pharmacokinetic Parameters of PEGylated rhG-CSF*

| | | PEG20-rhG-CSF | | PEG30-rhG-CSF | | PEG40-rhG-CSF | |
|---|---|---|---|---|---|---|---|
| Parameter | unit | 60 μg/kg | 100 μg/kg | 50 μg/kg | 100 μg/kg | 30 μg/kg | 60 μg/kg |
| Cₘₐₓ | ng/ml | 118.2 ± 75 | 196.7 ± 74 | 81.8 ± 93.8 | 122.9 ± 62.4 | 37.0 ± 16.1 | 83.6 ± 30.6 |
| Tₘₐₓ | hour | 15.0 ± 11.1 | 24.0 ± 7.0 | 24.0 ± 24.1 | 20.0 ± 13.9 | 13.5 ± 7.6 | 24.2 ± 18.5 |
| t_{1/2} | hour | 35.1 ± 8.4 | 41.0 ± 13.3 | 69.8 ± 33.5 | 70.2 ± 26.3 | 71.2 ± 30.2 | 75.5 ± 26.7 |
| AUC_{0-∞} | ng/h/ml | 5,610.6 ± 3,442 | 15,700.5 ± 8,432 | 4,073.9 ± 4,727 | 7,606.0 ± 3,343 | 2415.7 ± 930 | 5451.3 ± 1727 |
| CL/F | ml/h/kg | 20 ± 9.1 | 15 ± 6.7 | 30.0 ± 22.1 | 15.1 ± 7.0 | 20.7 ± 9.1 | 12.8 ± 5.3 |
| MRT | hour | 47.6 ± 13.2 | 53.5 ± 14.1 | 51.0 ± 13.9 | 46.3 ± 3.0 | 95.9 ± 18 | 83.5 ± 22 |
| I [symbol] < 2 [symbol] g/ml | hour | 216 | 216 | 144 | 168 | 288 | 336 |

Abbreviations: Cₘₐₓ, maximum observed plasma concentration; Tₘₐₓ, time of Cₘₐₓ; t_{1/2}, terminal half-life; AUC_{0-∞}, area under the plasma concentration-time curve from time zero to infinity; CL/F, apparent serum clearance after SC administration of dose; MRT, Mean residence time; T [symbol] < 2 ug/ml, time of plasma concentration less than 2 [symbol] g/nl which supposed to be as the lowest drug effective concentration. The T [symbol] < 2 [symbol] g/nl for the dose 12.5 μg/kg of PEG30-rhG-CSF is 72 hr.
*PEGylated rhG-CSF are FEG20-rhG-CSF(Neulasta), PEG30-rhG-CSF and PEG40-rhG-CSF, which are the N-terminally PEGylated rhG-CSF with different molecular weights such as 20-kd PEG moiety (PEG20), 30-kd PEG moiety (PEG30) ad 40-kd PEG moiety (PEG40) [symbol]
indicates data missing or illegible when filed

### 6. Composition and Formulation of N-Terminally Mono-Pegylated G-CSF

The composition and formulation of N-terminally mono-pegylated G-CSF, e.g., PEG30-rhG-CSF, comprises 10 mM-20 mM Sodium acetate, 0.004% Tween80 and 2.5%-5% Sorbitol, PEG30-rhG-CSF 1.5-6 mg/ml, and the pH is about 3.0-4.5.

7. The Effect of rhG-CSF, N- or C-Terminally Linked G-CSF on CXCR4 Expression of Stem Cells

As showed in FIG. 2, a combined Western Blot picture illustrated different effects of N-terminally or C-terminally linked G-CSF on CXC chemokine receptor-4 (CXCR4) expression in human CD33 bone marrow myeloid cells. G-CSF could inhibit the CXCR4 expression on stem cells, and so did the C-terminally linked G-CSF, e.g., GCSF-C-P20, GCSF-C-P30, GCSF-C-Fc and GCSF-C-HSA. The N-terminally linked G-CSF, e.g., P30-N-GCSF (PEG30-rhG-CSF), P40-N-GCSF, Fc-N-GCSF and HSA-N-GCSF could induce significantly the CXCR4 expression, P20-N-GCSF (Neulasta, PEG20-rhG-CSF) could partially induce the CXCR4 expression, but P10-N-GCSF couldn't induce the CXCR4 expression. Therefore, results indicated a macromolecular PEG polymer or fusion protein linked at N-terminal of rhG-CSF could significantly induce (or reduce the inhibition) the CXCR4 expression, and a molecular weight (MW) of conjugated PEG polymer or linked fusion proteins for the N-terminally linked G-CSF is equal to or more than the MW of no-glycosylated rhG-CSF, which is 18,800 Dalton (18.8 kDa).

8. The Effect of rhG-CSF, N- or C-Terminally Linked G-CSF on Mobilization of Progenitor Cells in Mice

BALB/c mice were intraperitoneally administered N-terminally linked G-CSF or C-terminally linked G-CSF (6 g or 12 g or 24 g/once/mouse) on Day 1 once, and administered rhG-CSF (0.3 g/day/mouse) daily for 5 days as positive control, or Normal saline daily for 5 days as negative control. Peripheral blood samples (PB) were collected 24 hours after the last injection of rhG-CSF, or on Day 6. PB was harvested from the orbital plexus into heparin-containing tubes. PB was diluted (1:4, v/v) with PBS, and mononuclear cells (MNCs) were separated by centrifugation (280×g, 30 min) on a Ficoll discontinuous density gradient. The MNCs were then washed twice in Iscove modified Dulbecco's medium supplemented with 10% FBS, 2 mM L-glutamine, and antibiotics.

Total CFCs (colony forming cells), i.e., CFU-GM, BFU-E, and CFU-GEMM were assessed in standard methylcellulose cultures. Briefly, lml aliquots of the MNCs (5×10⁴ to 2×10⁵ cells) were plated in 35-mm Petri dishes in methylcellulose-based medium (MethoCult™ GF M3434, STEMCELL Technologies, Canada). Colonies were scored after incubation at 37° C. in a humidified atmosphere of 5% CO₂ in air for 12-14 days. Data of total CFCs are presented in detail on Table 3.

TABLE 3
CFC counts in mice treated by rhG-CSF or
N-terminally or C-terminally linked G-CSF

| | CFCs/ml blood |
|---|---|
| Mobilization regimen (mice) | (mean ± SD) |
| Vehicle alone (n = 5) | 43 ± 7 |
| rhG-CSF (0.3 μg/day × 5) (n = 5) | 1258 ± 202 |
| P20-N-GCSF (6 μg once) (n = 5) | 579 ± 68^{a} |
| GCSF-C-P20 (6 μg once) (n = 5) | 2016 ± 268^{a} |
| P30-N-GCSF (6 μg once) (n = 5) | 226 ± 25^{b} |
| GCSF-C-P30 (6 μg once) (n = 5) | 3162 ± 412^{b} |
| Fc-N-GCSF (12 μg once) (n = 5) | 282 ± 39^{b} |
| GCSF-C-Fc (12 μg once) (n = 5) | 2687 ± 316^{a} |
| HSA-N-GCSF (24 μg once) (n = 5) | 239 ± 43^{b} |
| GCSF-C-HSA (24 μg once) (n = 5) | 1546 ± 458^{a} |
| P20-N-GCSF (1.5 μg Every-3-day ) (n = 5) | 186 ± 33 |
| P20-N-GCSF (0.6 μg daily) (n = 5) | 152 ± 28 |
| P30-N-GCSF (3 μg Every-3-day) (n = 5) | 183 ± 40 |
| P30-N-GCSF (1.0 μg daily) (n = 5) | 136 ± 35 |

^{a}p ≤ 0.05 as compared to rhG-CSF 5 μg/day for 5 days.
^{b}p ≤ 0.01 as compared to rhG-CSF 5 μg/day for 5 days.

As showed in Table 3, the total CFCs induced by the C-terminally linked G-CSF are 3.5 to 14 times greater than that of the N-terminally linked G-CSF. CFCs induced by the C-terminally linked G-CSF are also significantly greater than that of rhG-CSF, and CFCs induced by the N-terminally linked G-CSF are also significantly less than that of rhG-CSF. The results indicated there were negative correlation between the mobilizing effects of PBPC and CXCR4 expression levels on stem cells.

9. The Effect of N-Terminally or C-Terminally Linked G-CSF on Mobilization of Neutrophils in Rats with CIN

As showed in Table 4, comparing with the C-terminally linked G-CSF, the N-terminally linked G-CSF significantly reduced values of ANC release peak nearly two times, substantially elevated values of ANC nadir points 2.8 to 11 times, and significantly improve values of ANC recovery peak 1.4 to 3.6 times.

### 10. Characteristics of De-Mobilized G-CSF and High-Mobilized G-CSF

As showed in Table 3 & Table 4, the N-terminally linked G-CSF has shown obviously reduced the mobilization of neutrophils and progenitor cells, and amplified the granulopoiesis compared with rhG-CSF and C-terminally linked G-CSF. Therefore, the N-terminally linked G-CSF is a De-mobilized G-CSF. The characteristics of the De-mobilized G-CSF are summarized as follows: i) a PEG polymer conjugated or a Fc fragment or an albumin fusioned to the N-terminal of the G-CSF; ii) the molecular weight (MW) of the polymer or fusion protein is in range of 18,800-66,000 Dalton (19-66 kDa); iii) significantly increased CXCR4 expression comparing with G-CSF or C-terminally mono-pegylated G-CSF; iv) significantly decreased the values of neutrophils and CD34⁺ cells release peaks comparing with rhG-CSF and High-mobilized G-CSF, e.g., G-CSF-C-P30.

As showed in Table 3 & Table 4, the C-terminally linked G-CSF has shown obviously increased the mobilization of neutrophils and progenitor cells, and reduced the granulopoiesis compared with rhG-CSF and N-terminally linked G-CSF. The C-terminally linked G-CSF is a High-mobilized G-CSF. The characteristics of the High-mobilized G-CSF are summarized as follows: i) a PEG polymer conjugated or a Fc fragment or an albumin fusioned to the C-terminal of the G-CSF; ii) the MW of the polymer or fusion protein is in range of 19-66 kDa; iii) significantly inhibited CXCR4 expression compared with De-mobilized G-CSF; iv) significantly increased the values of neutrophils and CD34⁺ cells release peaks compared with rhG-CSF and De-mobilized G-CSF, e.g., P30-N-GCSF (PEG30-rhG-CSF).

### Example 2. Application of De-Mobilized G-CSF in Rats with Chemotherapy-Induced Neutropenia (CIN)

Sprague-Dawly Rats, male, 150-200 g, were obtained from Beijing Vital River Laboratory Animal Technology Co., Ltd. Rats were allowed acclimate for about 5 days before experiments. Rats were randomized to different test groups according to their Day 0 absolute neutrophil counts (ANC). There were seven rats in each of the test group. One experiment contains four to six test groups. In all but one of the test groups, rats were injected intraperitoneally 110 mg/kg of cyclophosphamide (CPA, Jiangsu Suncadia, China) on day 0 to induce neutropenia, the chemotherapy-induced neutropenia (CIN). One group of control rats did not receive CPA. The different test groups were received subcutaneous injections (s.c.) of PEG30-rhG-CSF (Tianjin Pegylatt, China), Neulasta (Amgen, USA), or Neupogen (Filgrastim, Amgen, USA), or formula buffer (10 mM Sodium acetate, 5% Sorbitol, pH 4.0), with different administration time, mode or dose according to study designs. The protein concentration of PEG30-rhG-CSF, Neulasta or Neupogen was measured by a modified Lowry's method as described in the Chinese Pharmacopoeia. The micro blood samples were collected from the tail vein using EDTA anticoagulation tubes on Day0, Day1-9, Day12 and Day15 in a chemotherapy cycle, and performed CBC analyses by Sysmex pocH-100i. Statistical analyses between groups or among the groups were done on parameters of ANC response curve (ANC-RC) by Student's t-test or Covariance analysis, and the statistical significance was set at p<0.05. The results of the preclinical studies were shown in Table 4 and FIGS. 3A-3H.

TABLE 4
Effect of N-terminally or C-terminally
linked G-CSF on ANC-RC in rats with CIN

| Administration Method | ANC release peak | ANC nadir point | ANC recovery peak |
|---|---|---|---|
| Once-per-cycle, on Day 2 | (cells/ml) | (cells/ml) | (cells/ml) |
| P20-N-GCSF (300 μg/kg) | 6.05 × 10⁶ | 0.52 × 10⁶ | 3.52 × 10⁶ |
| GCSF-C-P20 (300 μg/kg) | 10.8 × 10⁶ | 0.11 × 10⁶ | 0.98 × 10⁶ |
| P30-N-GCSF (300 μg/kg) | 5.08 × 10⁶ | 1.87 × 10⁶ | 3.93 × 10⁶ |
| GCSF-C-P30 (300 μg/kg) | 11.4 × 10⁶ | 0.17 × 10⁶ | 1.36 × 10⁶ |
| Fc-N-GCSF (600 μg/kg) | 7.38 × 10⁶ | 0.92 × 10⁶ | 5.32 × 10⁶ |
| GCSF-C-Fc (600 μg/kg) | 15.3 × 10⁶ | 0.14 × 10⁶ | 2.46 × 10⁶ |
| HSA-N-GCSF (1200 μg/kg) | 6.36 × 10⁶ | 0.36 × 10⁶ | 2.05 × 10⁶ |
| GCSF-C-HSA (1200 μg/kg) | 10.7 × 10⁶ | 0.13 × 10⁶ | 1.48 × 10⁶ |

1. Effect of Different Administration Times of PEG30-rhG-CSF or Neulasta on ANC Response Curves (ANC-RC) in Rats with CIN

As shown in FIG. 3A, two kinds of De-mobilized G-CSF, PEG30-rhG-CSF or Neulasta (Pegfilgrastim), were administered 300 μg/kg once per cycle on Day1 (D1) or Day 2 (D2). The injection time of D1 was same as 24 hours after the chemotherapy. The D1 injection time was also in the duration of neutrophils release peak induced by CPA, and D2 injection time was away from the period of the neutrophils release peak induced by chemotherapy drugs.

For D1 injection, the value of neutrophils release peak of PEG30-rhG-CSF or Neulasta was 5.46×10⁶ or 8.19×10⁶ cells/ml; the value of ANC nadir point was 0.6±0.2×10⁶ or 0.3±0.1×10⁶ cells/ml; the ratio of neutrophils release peak and nadir point (P/B ratio) was 9.1 and 27.3; and the value of neutrophils recovery peak was 3.6×10⁶ or 2.9×10⁶ cells/ml. For D2 injection, the value of neutrophils release peak was 4.96×10⁶ or 6.08×10⁶ cells/ml, and the value of ANC nadir point was 1.6±0.6×10⁶ or 0.8±0.2×10⁶ cells/ml. The P/B ratio was 3.1 or 7.6 and the value of neutrophils recovery peak were 4.3×10⁶ or 3.6×10⁶ cells/ml.

Standards of an effective and safe ANC response curve (esANC-RC) could be met by PEG30-rhG-CSF at dose of 300 μg/kg (equal to 50 μg/kg in human), once-per-cycle, on Day 2.

2. Effect of N-Terminally or C-Terminally Linked G-CSF on ANC-RC in Rats with CIN

As showed in Table 4, for Once-per-cycle mode, administration on Day 2 and same dose injected (equal to 50 μg/kg in human) by N-terminally or C-terminally linked G-CSF, values of ANC release peak induced by the N-terminally linked G-CSF are nearly two times less than that of the C-terminally linked G-CSF. Values of ANC nadir point induced by the N-terminally linked G-CSF are 2.8 to 11 times greater than that of the C-terminally linked G-CSF. The results indicated that the value of ANC nadir point could be elevated by reducing the value of ANC release peak, and a limited decrease of the value of ANC release peak can lead to several times increase of the value of ANC nadir point and recovery peak.

3. ANC-RC Induced by Injecting Neulasta Once on Different Times in Rats with CIN

As shown in FIG. 3B, ANC-RC was induced by injecting Neulasta at 600 μg/kg, once-per-cycle, on Day 2 (D2) or Day 3 (D3) of the chemotherapy cycle. For the injection on D2 or D3, the value of ANC nadir point was 1.1±0.3×10⁶ or 2.0±0.6×10⁶ cells/ml, the neutrophils release peak was 8.03±1.7 or 6.3±1.3×10⁶ cells/ml, the P/B ratio was 7.3 or 3.15. The Day 3 injection is three days before the ANC nadir point. Standards of esANC-RC could be achieved by injecting Neulasta on the Day 3, and a limited decrease of the ANC release peak can lead to several times increase of the value of ANC nadir point.

4. Effects of Different Dose-Intervals of Twice-Per-Cycle Mode for PEG30-rhG-CSF on ANC-RC in Rats with CIN

As shown in FIG. 3C, PEG30-rhG-CSF was administered 60 μg/kg for twice per cycle at different dose interval, e.g., Every-1-day on Day 2 and Day 3, Every-2-day on Day 2 and Day 4, Every-3-day on Day 2 and Day 5, or Every-4-day on Day 2 and Day 6. Results showed that values of neutrophil release peak were in turn 6.3±1.9, 5.2±1.7, 4.5±0.7, or 4.3±1.4×10⁶ cells/ml, ANC nadir points were in turn 1.1±0.3, 2.5±0.8, 2.7±0.3, or 1.3±0.4×10⁶ cells/ml, and P/B ratios were in turn 5.7, 2.1, 1.7 or 3.3. Therefore, the minimized fluctuation of ANC-RC could be received by the Every-3-day, the three days dosing interval. The minimized fluctuation of ANC-RC means the maximum of ANC nadir point, lower height of neutrophils release peak and minimum P/B ratio.

5. Effects of Every-3-Day and Once-Per-Cycle of Neulasta on ANC-RC in Rats with CIN

As shown in FIG. 3D, comparing with once-per-cycle mode at dosage 600 μg/kg, the value of neutrophil release peak and the P/B ratio for Every-3-day mode were significantly decreased (5.03±1.3×10⁶ vs. 8.03±1.7×10⁶, t test, p<0.05; 3.3 vs. 7.3, p<0.01), the value of ANC nadir point was significantly increased (1.9±0.5 vs. 1.1±0.5, t test, p<0.01). Standards of esANC-RC were met by administration 90 μg/kg (equal to 15 μg/kg in human) Every-3-day on Day 2 and Day 5 for Neulasta. The results indicated that there is more effective potential for the Every-3-day mode at 90 μg/kg twice in a cycle compared with the Once-per-cycle mode at 600 μg/kg (equal to 100 μg/kg in human) once in a cycle.

6. Effects of Every-3-Day and Once-Per-Cycle of PEG30-rhG-CSF on ANC-RC in Rats with CIN

As shown in FIG. 3E, standards of esANC-RC were met by the mode of Every-3-day at dosage of 60 μg/kg (equal to 10 μg/kg in human) and the mode of Once-per-cycle of PEG30-rhG-CSF at dosage of 600 μg/kg. However, the cycle dosage of the Every-3-day was 5 times less than that of the Once-per-cycle, the fluctuation of ANC-RC was more limited, and all of the key parameters of ANC-RC were in normal range of ANC.

The mode of Once-per-cycle could be replaced by Every-3-day mode with significantly reduced mobilization of neutrophils and increased the granulopoiesis. In clinical practice, the once-per-cycle mode applied in the previous chemotherapy cycle can be replaced by Every-3-day mode of De-mobilized G-CSF in present cycle when FN or grade 4 neutropenia occurred.

7. Effects of Every-Day and Once-Per-Cycle of PEG30-rhG-CSF on ANC-RC in Rats with CIN

As shown in FIG. 3F, for the Every-day mode of PEG30-rhG-CSF for 5 days at a dosage of 15 μg/kg/day (equal to 2.5 μg/kg/day in human), the value of neutrophils release peak was 4.6±0.9×10⁶ cells/ml, the value of ANC nadir point was 2.5±0.9×10⁶ cells/ml, and the P/B ratio was 1.4. Standards of esANC-RC were met by the Every-day mode at a dosage of 15 μg/kg/day for 5 days. For the Once-per-cycle mode of PEG30-rhG-CSF at a dosage of 150 μg/kg or 300 μg/kg, value of neutrophils release peak was 4.6±0.9 or 5.0±1.0×10⁶ cells/ml, ANC nadir point was 1.2±0.6 or 1.6±0.6×10⁶ cells/ml and P/B ratio was 3.8 or 3.1.

A key parameter of esANC-RC, the value of ANC nadir point, was significantly increased by Every-day mode 15 μg/kg/day for 5 days compared with that of Once-per-cycle 150 μg/kg or 300 μg/kg.

8. Effects of Every-Day and Every-3-Day of PEG30-rhG-CSF on ANC-RC in Rats with CIN

As shown in FIG. 3G, for the Every-day mode of PEG30-rhG-CSF at 30 μg/kg/day daily for 5 days or at 15 μg/kg/day for 10 days, the value of neutrophils release peak was 4.0±1.4×10⁶ or 3.6±0.9×10⁶ cells/ml, the value of ANC nadir point was 3.2±0.9×10⁶ or 2.5±0.9×10⁶ cells/ml, the P/B ratio was 1.25 or 1.44, the value of ANC recovery peak was 5.8±1.0×10⁶ or 3.5±1.1×10⁶ cells/ml. For the Every-3-day mode of PEG30-rhG-CSF at 150 μg/kg for 2 doses, the value of neutrophils release peak was 4.8±1.3×10⁶ cells/ml, the value of ANC nadir point was 2.2±0.9×10⁶ cells/ml, the P/B ratio was 2.2, and the value of ANC recovery peak was 4.3±1.4×10⁶ cells/ml. The standards of esANC-RC could be met by Every-day and Every-3-day mode at the dose selected. More limited mobilization and closer normal range of ANC was received by the Every-day mode at 15 μg/kg/day for 10 days. Similar results could be gotten by Every-day mode of Neulasta at the same dosage range, data not been provided. The results indicated that there is more effective potential for the Every-Day mode of De-mobilized G-CSF compared with the Every-3-Day mode.

There are evidences that the Every-day mode of De-mobilized G-CSF at 5 μg/kg/day for 5 days didn't promote the mobilization of mature neutrophils and hematopoietic progenitor cells or stem cells from bone marrow into peripheral blood (as shown in Table 3).

The mode of Every-3-day could be replaced by Every-day mode of the De-mobilized G-CSF with significantly reduced mobilization of neutrophils and stem cells, and significantly increased granulopoiesis. In clinical practice, the Every-3-day mode applied in previous chemotherapy cycle should be replaced by Every-day mode of De-mobilized G-CSF in present cycle when FN or Grade 4 neutropenia occurred.

9. Effects of Every-Day Mode of Neupogen and De-Mobilized G-CSF on ANC-RC in Rats with CIN

The effects of Every-day mode of rhG-CSF and De-mobilized G-CSF on ANC-RC in rats with CIN were illustrated in FIG. 3H. For Neupogen 50 μg/kg/day (equal to 8.3 μg/kg/day in human) for 10 days, the value of neutrophils release peak was 7.4±2.9×10⁶ cells/ml, the ANC nadir point was 2.5±0.5×10⁶ cells/ml, and the P/B ratio was 2.9. For PEG30-rhG-CSF or Neulasta 5 μg/kg/day for 10 days (equal to 0.83 μg/kg/day in human), the value of neutrophils release peak was 3.8±1.4×10⁶ or 4.4±0.9×10⁶ cells/ml, the ANC nadir point was 2.9±0.8×10⁶ or 2.1±0.5×10⁶ cells/ml, and the P/B ratio was 1.3 or 2.1. The results have showed that the efficacy of PEG30-rhG-CSF and Neulasta 5 μg/kg/day daily is equivalent with or even better than that of Neupogen 50 μg/kg/day daily injection in term of standards of esANC-RC. Results also indicated that Neupogen could be replaced by a De-mobilized G-CSF in daily administration mode for almost all of the indications of Neupogen approved except the mobilization of peripheral blood stem cells or progenitor cells (PBSC or PBPC).

10. Effects of Different Schedules of De-Mobilized G-CSF on Mortality Rates in Rats with CIN

To test effects of different administration methods of De-mobilized G-CSF on mortality rates in rats with CIN, all rats were injected intraperitoneally 110 mg/kg of CPA three doses on day 0, day 16 and day 32 for three chemotherapy cycles. There were twelve rats in each of test groups. One test group as a negative control, rats with CIN was injected the formula buffer. Rats in other test groups were injected PEG30-rhG-CSF or Neulasta using different methods according to the study design. Statistical analyses between groups were done on mortality rates using the Student's t-test, and the statistical significance was set at p<0.05. The results of the experiments were shown in Table 5.

TABLE 5
Effects of different schedules of De-mobilized
G-CSF on mortality rates in rats with CIN

| Administration Method | mortality rate |
|---|---|
| Rats injected CPA on Day 0, 16 & 32 | 66.7% |

Rats with CIN injected Neulasta for three cycles

| | |
|---|---|
| Once-per-cycle, 600 μg/kg, Day 2 | 100%^{a} |
| Once-per-cycle, 600 μg/kg, Day 3 | 66.7% |
| Every-3-day, 90 μg/kg, Day 2& Day 5 | 41.7%^{a} |
| Every-day, 5 μg/kg/day, Day 2-Day 11 | 25.0%^{b} |

Rats with CIN injected PEG30-rhG-CSF for three cycles

| | |
|---|---|
| Once-per-cycle, 300 μg/kg, 12 hours after chemotherapy | 100%^{a} |
| Once-per-cycle, 300 μg/kg, Day 2 | 41.7%^{a} |
| Every-3-day, 60 μg/kg, Day 2& Day 5 | 16.7%^{b} |
| Every-day, 15 μg/kg/day, Day 2-Day 6 | 16.7%^{b} |

^{a}p ≤ 0.05 as compared to mortality rate of the negative control group. . .
^{b}p ≤ 0.001 as compared to mortality rate of the negative control group.

As shown on Table 5, the mortality rates were significantly correlation with administration schedules of De-mobilized G-CSF in treatment of rats with CIN in successive three chemotherapy cycles. It is unexpected and surprised finding that not all administration schedules of De-mobilized G-CSF would be beneficial to the survival of rats with CIN, the approved schedule of Neulasta was even harmful to the survival of the rat with CIN, and so did the PEG30-rhG-CSF (Once-per-cycle, injected 300 μg/kg, at 12 hours after chemotherapy). The mortality rate by the approved schedule of Neulasta or PEG30-rhG-CSF was significantly increase compared with that of rats with CIN only. The results indicated the administration schedules of De-mobilized G-CSF that led to meet standards of esANC-RC would be beneficial to the survival of rats with CIN.

### Example 3 Application of De-Mobilized G-CSF in Macaques with CIN

Healthy macaques, 35, 5-9 kg, were obtained from Suzhou Xishan Zhongke Experimental Animal Co. Ltd. Macaques were allowed acclimate for about 7 days under GLP condition. Macaques were grouped according to the ANC on Day 0. There were 5 macaques per test group. All macaques in test groups were received 6 mg/m² dose of Chlormethine Hydrochloride (Shanghai Xudong Haipu, China) in 50 mL normal saline by intravenous drip. The animals in each of test groups were received De-mobilized G-CSF or formula buffer by subcutaneous injection once a cycle, or by subcutaneous, continuing controlled delivery (CCD) according to the study design. The CCD was performed by using DANA Insulin-Pump IIS (SOOIL, Korea) at a constant speed for lasting 3 or 5 days delivery (3-Day-CCD or 5-Day-CCD). The blood samples were collected from the tail vein using EDTA anticoagulation tubes on Day 0, Day 1-12 and Day14, 16, 18 and 21, and the CBC was performed by Sysmex XN-1000. The blood sample for count of periphery blood stem cells was collected after De-mobilized G-CSF injection on day 1, 3, 5, 7, 9, 11, 13 &15, and counts of CD34⁺ cells were determined by flow cytometric analysis. Statistical analyses between groups were done on parameters of ANC-RC by using Student's t test with significance set at p<0.05. The results of the experiment were shown in FIG. 4 and Table 6.

As shown in FIG. 4, standards of esANC-RC and sPBPC-RC were met by application of De-mobilized G-CSF with CCD mode at the dose 120 μg/kg for lasting 5 days. The administration time was 24 hours after chemotherapy (on Day 2). Almost all ANC values in cycle were kept in normal range of 2.5-6.5×10⁹ cells/L with reduced or even disappeared neutrophils release peak. No significant differences were found for the ANC release peak, the nadir point or the P/B ratio between PEG30-rhG-CSF and Neulasta. Standards of esANC-RC and sPBPC-RC were also met by the 3-5-day CCD mode of PEG30-rhG-CSF at the dosage range of 30 μg/kg-150 μg/kg, or the 7-day CCD mode of Neulasta at the dosage of 150 μg/kg as shown in Table 6.

As shown in FIG. 4, the ANC-RC induced by 3-day CCD of Neulasta with 120 μg/kg dosage delivered by insulin pump for three days couldn't meet standards of esANC-RC. The value of ANC release peak induced by the 3-day CCD mode and 120 μg/kg dose of Neulasta was 13.80±2.31×10⁹ cells/L, the ANC nadir point was 1.5±0.82×10⁹ cells/L, and the P/B ratio was 9.2. However, PEG30-rhG-CSF with 3-day CCD at the dosage of 120 μg/kg could achieve standards of esANC-RC.

As shown in FIG. 4, application of Once-per-cycle mode of Neulasta or PEG30-rhG-CSF with 300 μg/kg dosage failed to meet standards of esANC-RC. Compared with the Once-per-cycle of Neulasta or PEG30-rhG-CSF 300 μg/kg on Day 4, Neulasta or PEG30-rhG-CSF applied 5-day-CCD mode with 120 μg/kg on Day 2 reduced 4.47 or 2.11 times on the value of neutrophils release peak, elevated 36.21 or 8.95 times on the value of ANC nadir point, and dramatically decreased 161.42 or 18.79 times on the P/B ratio. Therefore, the CCD mode of De-mobilized G-CSF is more effective than that of Once-per-cycle mode.

Standards of sPBPC-RC could be met by application of 5-Day-CCD of PEG30-rhG-CSF with 150 μg/kg (equal to 50 μg/kg in human) or 7-Day-CCD of Neulasta with 150 μg/kg dosage as shown in Table 6.

TABLE 6
More results of continuing controlled delivery (CCD) by De-mobilized G-CSF (×10⁹ cells/L)

| | ANC | ANC | P/B | standards | standard |
|---|---|---|---|---|---|
| | release Peak | nadir point | ratio | esANC-RC | sPBPC-RC |
| PEG30-rhG-CSF on day 2/4 | (×10⁹ cells/L) | (×10⁹ cells/L) | | | |
| 15 μg/kg 3-Day-CCD D 2 | 4.34 ± 3.01 | 1.08 | 4.0 | no | yes |
| 30 μg/kg 5-Day-CCD D 2 | 5.12 ± 2.03 | 1.56 | 3.3 | yes | yes |
| 150 μg/kg 5-Day-CCD D 2 | 8.58 ± 3.56 | 4.23 | 2.0 | yes | yes |
| Neulasta on day 1/2/3/4 | (×10⁹ cells/L) | (×10⁹ cells/L) | | | |
| 30 μg/kg 3-Day-CCD D 2 | 5.73 ± 2.54 | 1.07 | 5.4 | no | yes |
| 30 μg/kg 5-Day-CCD D 1 | 5.59 ± 2.43 | 0.83 | 6.7 | no | yes |
| 150 μg/kg 7-Day-CCD D 3 | 6.76 ± 3.34 | 3.67 | 1.8 | yes | yes |

### Example 4 Application Neulasta or Neupogen in Patients with Less than 10% Risk of FN

A prospective, multi-center, randomized and open-label clinical study was performed to evaluate effects on ANC-RC for Once-per-cycle of Neulasta 50 μg/kg on Day 7 compared with 100 μg/kg on Day 3 (48 hours after chemotherapy) or Neupogen daily on Day 3. Neulasta was injected subcutaneously (SC) once on Day 7 or Day 3, or Neupogen was injected daily 5 μg/kg/day or 2.5 μg/kg/day for 10 days on Day 3. Patients with breast cancer received chemotherapy (AC regimen, Doxorubicin 60 mg/m² or Epirubicin 100 mg/m² and Cyclophosphamide 600 mg/m², intravenous infusion on Day 1). Patients were eligible for the study if they were ≥18 years of age, had a Karnofsky performance status ≥70%, ANCs ≥1.5×10⁹ cells/L, and platelet counts ≥100×10⁹ cells/L. Patients were excluded from the study if they had had previous systemic chemotherapy or extensive radiotherapy, had current uncontrolled infection, or had cancer other than breast cancer, or had bone marrow metastases. The complete blood cell counts (CBC) were performed daily from Day 0 to Day 9, and on Day 11, 13, 15, 17, 19 and Day21. The CBC was measured by Sysmex automated hematology analyzer, and the ANC between groups were compared using student t test (p<0.05 was set as the significant difference). The selected patients are assessed with less than 10% risk of febrile neutropenia (FN) and greater than 50% Grade 3/4 neutropenia.

As showed in FIG. 5, comparing Once-per-cycle of Pegfilgrastim 50 μg/kg on Day 7 with 100 μg/kg on day 3 on key parameters of ANC-RC for patients with less than 10% risk of FN, the value of neutrophils release peak (14.4×10⁹ vs. 32.1×10⁹ cells/L, t test, p<0.01) was significantly reduced, the value of ANC nadir point (4.7×10⁹ vs. 2.0×10⁹ cells/L, t test, p<0.01) was significantly increased, and there is more than five times decreased between the two P/B ratios (3.1 vs. 16). Standards of esANC-RC could be met by administrating Pegfilgrastim 50 μg/kg once on Day 7, and no incidence of Grade 3/4 neutropenia was occurred, but not 100 μg/kg once on Day 3. The Day 7 of chemotherapy cycle is four days before the date of ANC nadir point.

As shown in FIG. 5, for the Every-day mode of Filgrastim injected on Day 3, comparing 2.5 μg/kg/day daily for 10 days with 5 μg/kg/day for 10 days, the value of ANC release peak (17.6×10⁹ vs. 23.2×10⁹ cells/L, t test, p<0.05) and the value of neutrophils recovery peak (11.5±4.7×10⁹ vs. 27.4±15×10⁹ cells/L, t test, p<0.01) were significantly decreased. No significant different between the values of ANC nadir point (5.7±2.5×10⁹ vs. 7.0f8.1×10⁹ cells/L, t test, p>0.05) were found. Therefore, for those patients, standards of esANC-RC could be achieved by application of Filgrastim 2.5 μg/kg/day for 10 days, not 5 μg/kg/day for 10 days.

Key parameters of ANC-RC induced by the approved mode, dose and time of Neulasta or Neupogen were far inferior to standards of esANC-RC in the selected patients.

As shown in FIG. 5, the approved method of Neulasta for the selected patients can lead to the highest value of neutrophils release peak with the lowest value of ANC nadir point. The extraordinary high release peak of neutrophils resulted from accelerating clearance of mature neutrophils in bone marrow, and could accelerate the eliminating of Pegfilgrastim, and resulted in lower value of ANC nadir point. The results indicated that to control the neutrophils releasing could significantly increase the value of ANC nadir point.

### Example 5 Application PEG30-rhG-CSF or Neupogen in Patients with about 20% Risk of FN

As shown in FIG. 6A, 6B, 6C, PEG30-rhG-CSF was evaluated in a single-center, open-label, dose-escalation clinical study. Patients with pathological diagnosed non-small cell lung cancer (NSCLC) were received chemotherapy of TC regimen (Taxol 175 mg/m² and Carboplatin AUC 6 on Day 1). Patients who were eligible for or excluded from the study were as same as Example 4. PEG30-rhG-CSF or Filgrastim was administered on Day 5, seven days before the date of ANC nadir point in the chemotherapy cycle. In cycle 1, there were five sequentially enrolled PEG30-rhG-CSF cohorts (dose levels of 12.5, 25, 50, 75 and 100 μg/kg). In cycle 2, there were three sequentially enrolled Filgrastim cohorts (dose levels of 1.25, 2.5 and 5 μg/kg/day for ten days). Patients receiving 12.5, 25, 50, 75 or 100 μg/kg of PEG30-rhG-CSF in Cycle 1 would be applied by Filgrastim 1.25, 2.5, 5, 5, 5 μg/kg/day daily for ten days in cycle 2. Neulasta was administered on Day 3, nine days before the date of ANC nadir point in the chemotherapy cycle. There were two sequentially enrolled Neulasta cohorts (dose levels of 60 and 100 μg/kg). The complete blood cell counts (CBC) were performed daily from Day 0 to Day 13, and on Day 15, 17, 19 and Day 21. The CBC was measured by Sysmex automated hematology analyzer. CD34⁺ cell counts were determined on every other day from Day 5-Day 15 and Day 21 by the flow cytometric analysis. Statistical analyses between groups or among the groups were done on parameters of ANC-RC and PBPC—RC by Student's t-test or Covariance analysis, and the statistical significance was set at p<0.05. The selected patients are assessed with about 20% risk of FN and with about 100% risk of Grade 3/4 neutropenia.

As shown in FIG. 6A, for application of PEG30-rhG-CSF 12.5 μg/kg, 25 μg/kg, 50 μg/kg, 75 μg/kg or 100 μg/kg once on Day 5, the value (Mean+SD) of ANC release peak for each of groups was 18.60±3.86, 17.92±11.75, 22.05±6.18, 19.28±6.03 or 21.37±5.15×10⁹ cells/L; the value of ANC nadir point was 1.60±1.23, 1.39±0.98, 2.34±1.52, 5.06±2.47 or 8.51±6.0×10⁹ cells/L; the P/B ratio was 11.65, 12.89, 9.0, 3.8 or 2.6; and the value of ANC recovery peak was 6.37±2.06, 4.72±2.68, 6.89±3.51, 8.97±4.37 or 16.79±6.87×10⁹ cells/L. Key parameters of ANC-RC achieved by injection 50 μg/kg or 75 μg/kg of PEG30-rhG-CSF were closer to meet standards of esANC-RC.

As shown in FIG. 6B, for application of Filgrastim 2.5 μg/kg/d or 5 μg/kg/d daily for 10 days, the value of ANC release peak was 10.35±3.77 or 13.17±3.0×10⁹ cells/L, the value of ANC nadir point was 3.54±2.50 or 4.76±2.06×10⁹ cells/L, the P/B ratio was 2.9 or 2.5, and the value of neutrophil recovery peak was 13.54±2.98 or 16.34±6.90×10⁹ cells/L. Key parameters of ANC-RC achieved by daily injection 2.5 μg/kg/d of Filgrastim were closer to meet standards of esANC-RC.

In order to meet standards of esANC-RC, all clinical studies were performed with ANC determined on key time points of chemotherapy cycle, and an adjustable dosage or time or both was applied to the patient according to the ANC determined on the planned injection time. Results from two groups of this clinical study were provided.

Results from a group with adjustable dosage only were as follows: For Once-per-cycle, PEG30-rhG-CSF was planned to inject on Day 2 (24 hours after the chemotherapy), the administration cycle-dosage was 40 μg/kg if the ANC determined on Day 2 is in the range of 1.5-8.0×10⁹ cells/L. The cycle-dosage was decreased to 30 μg/kg if the ANC determined on Day 2 was more than 8.0×10⁹ cells/L; the cycle-dosage was increased to 50 μg/kg, if the ANC determined on Day 2 was lower than 1.5×10⁹ cells/L. A preliminary data from this group showed that the value of neutrophils release peak was 14.27±6.23×10⁹ cells/L; value of the ANC nadir point was 2.45±1.17×10⁹ cells/L; the P/B ratio was 5.82; and the value of neutrophil recovery peak was 3.61±2.75×10⁹ cells/L. Almost all of standards of esANC-RC except the value of neutrophils release peak were achieved by this adjustable dosage of 40 μg/kg of PEG30-rhG-CSF on Day 2.

Results from a group with adjustable time and dosage were as follows:

For Once-per-cycle, PEG30-rhG-CSF was planned to inject on Day 5, and administration cycle-dosage was 25 μg/kg if the ANC determined on Day 5 is in the range of 1.5-4.0×10⁹ cells/L. The injection time was postponed until the ANC drops to the range of 1.5-4.0×10⁹ cells/L, if the ANC determined on Day 5 was more than 4.0×10⁹ cells/L; the cycle-dosage was increased to an upper dose level of 50 μg/kg, if the ANC determined on Day 5 was lower than 1.5×10⁹ cells/L; the dosage was decreased to a lower dose level of 12.5 μg/kg, if the ANC determined on Day 5 was higher than 8.0×10⁹ cells/L and/or the administration time was postponed more than 4 days until the ANC drops to the range of 1.5-4.0×10⁹ cells/L. A preliminary data from this group showed that the value of neutrophils release peak was 12.35±3.31×10⁹ cells/L; value of the ANC nadir point was 4.36±1.22×10⁹ cells/L; the P/B ratio was 2.9; and the value of neutrophil recovery peak was 6.32±3.24×10⁹ cells/L. Standards of esANC-RC and sPBPC-RC could be fully achieved by this adjustable time and dosage of 25 μg/kg of PEG30-rhG-CSF on Day 5.

As shown in Table 7, there were close relationship between clinical efficacy and key parameters of ANC-RC and PBPC-RC. Compared with standards of esANC-RC and sPBPC-RC, higher value of neutrophils release peak, lower value of ANC nadir point, greater P/B ratio and higher value of CD34⁺ cell release peak could result in higher incidences of neutropenia, Grade 4 neutropenia and FN, or rate of anti-infective use. Standards of esANC-RC and sPBPC-RC could be met by the adjustable time and dosage of 25 μg/kg PEG30-rhG-CSF once-per-cycle on day 5, which are also free of FN and Grade 3/4 neutropenia and no anti-infective use.

Key parameters of ANC-RC induced by the approved mode, dose and time of Neulasta or Neupogen were far inferior to standards of esANC-RC in the selected patients.

TABLE 7
Clinical results of PEG30-rhG-CSF, Filgrastim and Pegfilgrastim in patients with CIN

| | PEG30-rhG-CSF(A) | | Filgrastim(B) | | | Pegfilgrastim(C) | | |
|---|---|---|---|---|---|---|---|---|
| | 25 μg/kg, once-per-cycle, | | 2.5 μg/kg/d, daily, | | A vs. B | 100 μg/kg, once-per-cycle, | | A vs. C |
| | at Day 5^{★} | | start at Day 5 | | t test | at Day 2^{∘} | | t test |
| Neutrophil release Peak | 12.35 ± 3.31 | | 10.35 ± 3.77 | | p < 0.01 | 33.6 ± 10.6 | (×10⁹/L) | p < 0.01 |
| ANC nadir point | 4.36 ± 1.22 | | 3.54 ± 2.7 | | p < 0.01 | 2.0 ± 0.6 | (×10⁹/L) | p < 0.01 |
| P/B ratio^{♦} | 2.9 | | 2.9 | | | 16.8 | | |
| Neutrophil recovery Peak | 6.32 ± 3.24 | | 13.54 ± 3.0 | | p > 0.05 | 11.59 ± 4.4 | (×10⁹/L) | p > 0.05 |
| CD34⁺ Cells release Peak | 0.72 ± 0.39 | | 6.0 ± 3.4 | | p > 0.05 | 43.0 ± 34.3 | (×10⁶/L) | p < 0.01 |
| | n | (%) | n | (%) | | n | (%) | |
| ANC nadir < 1.5 × 10⁹/L | 0 | (0) | 11 | (9.3%) | p < 0.05 | 70 | (53.0%) | p < 0.01 |
| ANC nadir < 0.5 × 10⁹/L | 0 | (0) | 3 | (1.8%) | p > 0.05 | 22 | (16.7%) | p < 0.01 |
| Febrile neutropenia | 0 | (0) | 0 | (0) | p = 1 | 5 | (3.8%) | p < 0.05 |
| Anti-infective use | 0 | (0) | 1 | (1.5%) | p > 0.05 | 45 | (34.1%) | p < 0.01 |

^{★}The Day 5 of the chemotherapy cycle is about seven days before the date of ANC nadir point.
^{∘}The Day 2 of the chemotherapy cycle is the 24 hours after the completion of chemotherapy.
^{♦}P/B ratio is the ratio of the neutrophil release peak to ANC nadir point.

As shown in FIG. 6C, for Once-per-cycle injection of PEG30-rhG-CSF 12.5 μg/kg, 25 μg/kg, 50 μg/kg or 100 μg/kg on Day 5, the values of CD34⁺ cells release peak were 1.93±0.71, 0.68±0.36, 0.76±0.58, or 6.87±1.04×10⁶ cells/L. For daily injection of Filgrastim 1.25 μg/kg/d, 2.5 μg/kg/d or 5 μg/kg/d for 10 days, the values of CD34⁺ cells release peak were 7.34±1.35, 5.97±3.38, or 9.59±1.55×10⁶ cells/L. Although the values of CD34⁺ cells release peaks were not totally dose-dependent, the value of CD34⁺ cells release peak for injection 100 μg/kg once of PEG30-rhG-CSF is nearly equal to that of daily injection 1.25 μg/kg/d or 2.5 μg/kg/d of Filgrastim. In general, values of CD34⁺ cells release peak induced by injection equal to or less than 50 μg/kg of PEG30-rhG-CSF could meet standards of sPBPC-RC. The CD34⁺ cells count of the preferable safe peak value is equal to or less than 1×10⁶ cells/L.

As shown in FIG. 6D, the value of CD34⁺ release peak induced by injection 60 μg/kg or 100 μg/kg Neulasta on Day 3 was 58.18±28.31 or 45.98±33.25×10⁶ cells/L. The value of CD34⁺ release peak induced by injection 50 μg/kg of PEG30-rhG-CSF was 0.77±0.58×10⁶ cells/L, which was 75 times lower than that of 60 μg/kg dosage of Neulasta. The value of CD34⁺ release peak induced by injection 100 μg/kg of PEG30-rhG-CSF was 6.87±1.04×10⁶ cells/L, which was nearly 7 times lower than that of injection 100 μg/kg of Neulasta, and was also significantly lower than that of Filgrastim at a dosage of 5 μg/kg/d (9.59±2.9×10⁶ cells/L). Results indicated that PEG30-rhG-CSF significantly reduced the mobilization of stem cells into peripheral blood.

As shown in FIG. 6E, for application of PEG30-rhG-CSF (100 μg/kg, once, on Day 5) or Neulasta (100 μg/kg, once, on Day 3), the value of neutrophils release peak was 21.4±0.52×10⁹ cells/L or 33.6±10.6×10⁹ cells/L, there are 1.6 times indifference; the value of ANC nadir point was 8.5±6.0×10⁹ cells/L or 2.0±0.55×10⁹ cells/L, there are 4.3 times in difference; the P/B ratio was 2.5 or 16.8, there are 6.7 times in difference. The results have demonstrated that a limited decrease value of the ANC release peak can lead to several times increase on the value of ANC nadir point.

As shown in FIGS. 6D & 6E, the PEG30-rhG-CSF could greatly reduce the mobilization of neutrophils and stem cells from bone marrow into peripheral blood. Therefore, compared with Neulasta, PEG30-rhG-CSF could effectively protect the hematopoietic functions of bone marrow for patients with solid tumor receiving chemotherapy, and accelerate the granulopoiesis of G-CSF.

The key parameter of PBPC-RC induced by the approved mode, dose and time of Neulasta or Neupogen was far more than standards of sPBPC-RC in the selected patients.

### Example 6 Application Every-3-Day Mode of PEG30-rhG-CSF in Patients with about 40% Risk of FN

As shown in FIG. 7, PEG30-rhG-CSF was evaluated in a multicenter, randomized, active-control clinical study. Patients who were eligible for study or excluded from the study were as same as Example 4. Patients with breast cancer were received chemotherapy of Docetaxel and Doxombicin regimen (Docetaxel 75 mg/m² and doxorubicin 60 mg/m²). Patients were randomized to receive PEG30-rhG-CSF with Every-3-day mode for twice or three times, or Neulasta with Once-per-cycle, or Filgrastim with Every-day mode in a chemotherapy cycle. PEG30-rhG-CSF with single-dose 25 μg/kg is administered for two times on Day 3 and Day 6, or for three times on Day 3, Day 6, and Day 9 if the ANC determined on Day 9 was lower than 1.5×10⁹ cells/L. Neulasta with cycle-dosage 100 μg/kg once or Neupogen with 5 μg/kg/d for ten days was applied on Day 2 as the positive-controls. The selected patients are assessed with about 40% risk of FN and with about 100% risk of Grade 3/4 neutropenia. The complete blood cell counts (CBC) were performed daily from Day 0 to Day 13, and on Day 15, 17, 19 and Day 21. The CBC was measured by Sysmex automated hematology analyzer. CD34⁺ cell counts were determined on every other day from Day 5-Day 15 and Day 21 by the flow cytometric analysis.

For the Every-3-day of PEG30-rhG-CSF injected 25 μg/kg for 2-3 doses, the value of ANC nadir point was 2.3±0.4×10⁹ cells/L, the value of ANC release peak was 14.6±5.8×10⁹ cells/L, the P/B ratio was 6.3, and the value of CD34⁺ cells release peak was less than 1×10⁶ cells/L (data not provided). Standards of esANC-RC and sPBPC-RC were achieved by this selected administration mode, dose and time of PEG30-rhG-CSF. No incidence of FN and Grade 3/4 neutropenia was occurred.

For the approved mode, dose and time of administration of Neulasta or Neupogen, value of the ANC nadir point was 0.05±0.02×10⁹ or 0.50±0.20×10⁹ cells/L, value of the ANC release peak was 27.4±7.2×10⁹ or 26.5±5.5×10⁹ cells/L, the P/B ratio was 548 and 53, and value of the neutrophil recovery peak was as 6.2±2.0×10⁹ or 28.5±10.3×10⁹ cells/L. Key parameters of ANC-RC achieved by the approved administration mode, dose and time of Neulasta or Neupogen were far inferior to standards of esANC-RC.

In summary, for all the selected patients in clinical study, key parameters of ANC-RC and PBPC-RC induced by the approved mode, dose and time of Neulasta or Neupogen were far inferior to standards of esANC-RC and sPBPC-RC; standards of esANC-RC and sPBPC-RC could be achieved by the Once-per-cycle or the Every-3-days modes with a new selected time and a controlled dosage of a De-mobilized G-CSF, and no incidence of FN and Grade 3/4 neutropenia was occurred in whole of chemotherapy cycle.

1. A method for treating or preventing febrile neutropenia (FN) or Grade 3/4 neutropenia comprising:
a) measuring the absolute neutrophils counts (ANC) and CD34⁺ cells counts in the peripheral blood (PBPC) of a cancer patient undergoing a chemotherapy to obtain a response curve of neutrophils (ANC-RC) or progenitor cells (PBPC-RC) in a chemotherapy cycle, and
b) injecting to the patient a long-acting granulocyte colony-stimulating factor (“De-mobilized G-CSF”) at an administration time and a dosage calculated based on classified risk of FN and/or the ANC determined on the planned injection day, to achieve an effective and safe ANC-RC and safe PBPC-RC,
wherein the patient suffers from chemotherapy-induced neutropenia (CIN) with an assessed risk of FN.

2. The method of claim 1, wherein the administration times comprises on the 7-9 days, or 5-7 days, or 3-5 days before the date of ANC nadir point for patient with CIN who has classified risks of greater than 40%, or a range of 10%-40%, or less than 10% risk of FN.

3. The method of claim 1, wherein the administration time is at the same day when the ANC determined on the planned injection day is lower than 4.0×10⁹ cells/L, or the administration time is postponed to 2 to 5 days until the ANC dropped to a range of 1.5-4.0×10⁹ cells/L when the ANC determined is higher than 4.0×10⁹ cells/L.

4. The method of claim 1, wherein the dosage is not more than 50 μg/kg of cycle-dosage, preferably not more than 25 μg/kg for patient with CIN who has 10%-40%, preferably not more than 20% risk of FN, and injected once in a cycle, the mode is Once-per-cycle.

5. The method of claim 1, wherein the dosage is in a range of 10 μg/kg/dose-25 μg/kg/dose of single-dose for 2-3 doses in a cycle for patient with CIN who has not more than 40% risk of FN, and the dosing interval is three days, the mode is Every-3-day.

6. The method of claim 1, wherein the dosage is in a range of 0.83 μg/kg/day-5 μg/kg/day of single-dose for patient with CIN who has greater than 40% risk of FN, and injected daily for 3-10 days in a cycle, the mode is Every-day.

7. The method of claim 1, wherein the dosage is in a range of 10 μg/kg-50 μg/kg of cycle-dosage, and the delivery rate of the dosage is continuously controlled for lasting 3-7 days for patient with CIN who has greater than 20% risk of FN, the mode is Continuing controlled delivery (CCD).

8. The method of any of claim 4, wherein the modes display an ascending order of efficacy from Once-per-cycle, Every-3-day and Every-day to CCD on the same cycle-dosage, and a more effective mode can be applied for present chemotherapy cycle when FN or Grade 4 neutropenia occurred in the previous chemotherapy cycle.

9. The method of claim 1, wherein the dosage is administered at an upper dose level of the mode if the ANC determined on the planned injection day is lower than 1.5×10⁹ cells/L, and the dosage is in a range of 10 μg/kg-75 μg/kg or 0.6 mg-4.5 mg of the cycle-dosage.

10. The method of claim 1, wherein the dosage is administered at a lower dose level of the mode if the ANC determined on the planned injection day is higher than 8.0×10⁹ cells/L and/or if the administration time has been postponed for more than 4 days, and the dosage is in a range of 5 μg/kg-50 μg/kg or 0.3 mg-3.0 mg of the cycle-dosage.

11. The method of claim 1, wherein the long-acting granulocyte colony-stimulating factor is identified as a mobilizing-declined G-CSF (De-mobilized G-CSF), and a kind of N-terminally linked G-CSF.

12. The method of claim 1, wherein the De-mobilized G-CSF is characterized by: i) a polyethylene glycol (PEG) polymer conjugated or a Fc fragment or an albumin fused or conjugated to the N-terminal of the G-CSF; ii) the molecular weight (MW) of the polymer or fusion protein is in range of 18,800-66,000 Dalton (18.8-66 kDa); iii) significantly increased CXCR4 expression comparing with G-CSF or C-terminally linked G-CSF; iv) significantly decreased the values of neutrophils and CD34⁺ cells release peaks comparing with rhG-CSF and C-terminally linked G-CSF, e.g., G-CSF-C-P30.

13. The method of claim 1, wherein the De-mobilized G-CSF comprises PEG20-rhG-CSF, PEG30-rhG-CSF, PEG40-rhG-CSF, Fc fusion G-CSF, or albumin fusion G-CSF.

14. The method of claim 1, wherein one of the De-mobilized G-CSF is a N-terminally mono-pegylated G-CSF, a PEG polymer conjugated at the N-terminal of G-CSF, including Pegfilgratim and PEG30-rhG-CSF.

15. The method of claim 1, wherein the G-CSF comprises G-CSF fragments or G-CSF mutants, the G-CSF mutants comprises one or more amino acid additions, deletions and/or substitutions, or small peptide fusion, and property changes of the G-CSF fragments or mutants comprise the increased solubility, stability, activity of granulopoiesis or half-life, or decreased activity of mobilization, as compared to that of the G-CSF.

16. A method for preparing a site-specific mono-pegylated G-CSF comprising: i) mixing a cosolvent to assist G-CSF solution concentrated to be more than 4 mg/ml; ii) adding an activated PEG polymer to the G-CSF solution, wherein the molar ratio of the PEG polymer to the G-CSF is in a range of 0.7-3 times, and under pH 3.5-5.0 or 7.0-8.5 for N-terminally or C-terminally site-specific PEG conjugation; iii) controlling reaction time by monitoring a formation rate of a di-pegylated G-CSF; wherein more than 95% of the site-specific mono-conjugated G-CSF is homogenously N-terminally or C-terminally mono-pegylated G-CSF.

17. The method of claim 16, wherein the cosolvent is a non-activated PEG polymer, Bicine or sorbitol.

18. The method of claim 16, wherein the reaction time is monitored by high performance gel chromatography to control the formation rate of di-pegylated G-CSF less than 5% in conjugates mixer.

19. The method of any of claim 5, wherein the modes display an ascending order of efficacy from Once-per-cycle, Every-3-day and Every-day to CCD on the same cycle-dosage, and a more effective mode can be applied for present chemotherapy cycle when FN or Grade 4 neutropenia occurred in the previous chemotherapy cycle.

20. The method of any of claim 6, wherein the modes display an ascending order of efficacy from Once-per-cycle, Every-3-day and Every-day to CCD on the same cycle-dosage, and a more effective mode can be applied for present chemotherapy cycle when FN or Grade 4 neutropenia occurred in the previous chemotherapy cycle.
